(19) United States
(12) Patent Application Publication
(10) Pub. No.: US 2023/0301914 A1
(43) Pub. Date: Sep. 28, 2023

(54) PHARMACEUTICAL COMPOSITIONS IN LYOPHILIZED FORM

(71) Applicant: Breath Therapeutics GmbH, Munich (DE)

(72) Inventor: Oliver Denk, Muensing (DE)

(21) Appl. No.: 18/317,940

(22) Filed: May 16, 2023

Related U.S. Application Data
(63) Continuation of application No. 17/251,223, filed on Dec. 11, 2020.

(30) Foreign Application Priority Data
Jun. 27, 2018 (EP) 18180177.0

(51) Int. Cl. A61K 9/127; A61K 9/00; A61K 9/19; A61K 38/13; A61K 47/24; A61K 47/26

(52) U.S. Cl. CPC A61K 9/127; A61K 9/0078; A61K 9/19; A61K 38/13; A61K 47/24; A61K 47/26

(57) ABSTRACT

The present invention relates to a lyophilized pharmaceutical composition for reconstitution in an aqueous carrier liquid comprising: a) liposome-forming structures comprising i. a therapeutically effective amount of cyclosporine A (CsA); ii. a membrane forming substance selected from the group of phospholipids; and iii. a solubility enhancing substance selected from the group of non-ionic surfactants; and b) at least one disaccharide selected from the group consisting of saccharose, lactose and trehalose, wherein the at least one disaccharide is present in an amount of at least 40 wt.-% with regard to the total weight of the lyophilized composition.

### FIELD

The present invention relates to a lyophilized pharmaceutical composition for reconstitution in an aqueous carrier comprising cyclosporine A (CsA) in liposomally solubilized form and at least one disaccharide selected from the group consisting of saccharose, lactose and trehalose.

The present invention also relates to liquid pharmaceutical preparations made by reconstitution of the lyophilized pharmaceutical compositions containing cyclosporine A in liposomally solubilized form as the active agent as well as substances which are especially suitable for pulmonary application. Further aspects of the invention relate to processes for the preparation of such compositions as well as to kits comprising said compositions. Furthermore, the invention relates to the pharmaceutical uses of the compositions and their application for the treatment of specific diseases such as pulmonary diseases.

### BACKGROUND

Cyclosporine (or ‘ciclosporin’ as used herein synonymously) is a cyclic oligopeptide with immunosuppressive and calcineurin inhibitory activity. It is characterised by a selective and reversible mechanism of immunosuppression by blocking the activation of T-lymphocytes by the production of certain cytokines which are involved in the regulation of these T-cells. This involves, in particular, the inhibition of the synthesis of interleukin-2 which, at the same time, suppresses the proliferation of cytotoxic T-lymphocytes which are responsible, for example, for the rejection of extraneous tissues. Cyclosporine acts intracellularly by binding to the so-called cyclophilines or immunophilines which belong to the family of proteins which bind cyclosporine with high affinity. The complex of cyclosporine and cyclophilin subsequently blocks the serine-threonine-phosphatase-calcineurin. Its activity state in turn controls the activation of transcription factors such as NF-KappaB or NFATp/c which play a decisive role in the activation of various cytokine genes including interleukin-2. This results in the arrest of the immunocompetent lymphocytes during the GO or G1 phase of the cellular cycle since the proteins which are essential for cell division such as interleukin-2 can no longer be produced. T-helper cells which increase the activity of cytotoxic T-cells which are responsible for rejection are the preferred site of attack for cyclosporine. Furthermore, cyclosporine inhibits the synthesis and release of further lymphokines which are responsible for the proliferation of mature cytotoxic T- lymphocytes and for other functions of the lymphocytes. The ability of cyclosporine to block interleukin-2 is critical for its clinical efficacy: transplant recipients which tolerate their transplants well are characterised by a low production of interleukin-2. Patients with manifest rejection reactions, on the contrary, show no inhibition of interleukin-2 production.

The first and so far only cyclosporine which has been placed on the market (in the 1980s) is cyclosporine A (CsA). CsA is defined chemically as cyclo-[[(E)-(2S,3R,4R)-3-hydroxy-4-methyl-2-(methylamino)-6-octenoyl]-L-2-aminobutyryl-N-methylglycyl -N-methyl-L-leucyl-L-valyl-N-methyl-L-leucyl-L-alanyl-D-alanyl-N-methyl-L-leucyl-N-methyl-L-leucyl-N-methyl-L-valyl]. Its availability initiated a new era in transplant medicine because, with its help, the proportion of transplanted organs which remain functional in the long term, could be increased substantially. The first cyclosporine medicament (Sandimmun® of Sandoz) could already increase the success rate in kidney transplantations by a factor of about 2. A new oral preparation of cyclosporine (Neoral of Sandoz, later Novartis) with higher and more reliable bio-availability allowed better dosing and further increase of the success rate since the 1990s. Despite some new developments of active agents, CsA is still a frequently used agent in transplantation medicine.

Today, lung transplantations can, in principle, also be carried out successfully if patients are treated with CsA. Since the introduction of this active agent in clinical therapy, the number of lung transplantations carried out worldwide has increased dramatically. This is true for both, the transplantation of a single lung as well as the transplantation of both lungs. Lung transplantations are normally contemplated in the case of patients with a final-staged lung disease where medicinal therapy has failed and life expectancy is short due to the disease. Transplantations of a single lung are indicated, for example, in the case of certain forms of emphysema and fibrosis, such as idiopathic pulmonary fibrosis. Both lungs are transplanted in cases of cystic fibrosis (mucoviscidosis), primary pulmonary hypertension, emphysema with global insufficiency, frequent serious infections as well as idiopathic pulmonary fibrosis with complication by repeated infections. In the case of a successful lung transplantation, the patients' quality of life can be increased again to an almost normal level. However, contrary to heart, kidney and liver transplantations, the survival times after lung transplantations are still relatively short and amount to an average of only 5 years. This might be due, amongst other things, to the fact that the active agent cyclosporine cannot be effectively dosed with all patients due to systemic side effects such as renal dysfunction, increased serum levels of creatinine and urea, renal damage with structural changes, for example, interstitial fibrosis, increased serum levels of bilirubine and liver enzymes, hypertrichiosis, tremor, fatigue, headache, gingivitis hypertrophicans, gastrointestinal complains like anorexia, abdominal pain, nausea, vomiting, diarrhoea, gastritis, gastroenteritis, paraesthesia, stinging sensations in the hands and feet, arterial hypertension, increased blood fat levels, acne, rashes, allergic skin reactions, hyperglycaemia, anaemia, hyperuricaemia, gout, increasing body weight, oedemas, stomach ulcers, convulsions, menstrual disorders, hyperkalaemia, hypomagnesaemia, hot flushes, erythema, itching, muscular cramps, muscular pain, myopathy, etc.

Therefore, it would be desirable, if, for example, after a lung transplantation or in cases of certain other indications, CsA could be administered in a targeted and tissue specific fashion and so as to achieve only a low systemic bioavailability of the active agent in order to minimize the impact of the active agent in healthy tissue.

A suitable dosage form could also be used for the treatment and prevention of diseases such as asthma, idiopathic pulmonary fibrosis, sarcoidosis, alveolitis and parenchymal lung diseases (see: Drugs for the treatment of respiratory dis-eases, edited by Domenico Spina, Clive p. Page et. al., Cambridge University Press, 2003, ISBN 0521773210). New therapeutic aspects also result for the topical treatment of possible autoimmune included diseases such as neurodermatitis, psoriasis, unspecific eczema, skin proliferations or mutations, and for the treatment after skin transplantations. An interesting area of application is in the field of ophthalmology, for example, for the treatment after corneal transplants, of keratoconjunctivitis or other infectious eye diseases which respond partly insufficiently to anti-inflammatory therapy, for example with steroids. It is also useful for the treatment of keratitis in animals, such as dogs.

Attempts have been made to administer cyclosporine locally, for example, in the form of oily eye drops at 1% and 2% (formulation according to the German codex of medicines using refined peanut oil as solubilizer) or as an aerosol. However, this approach normally fails, mainly due to the very low aqueous solubility of the active agent which renders efficient administration considerably difficult. Thus, in the case of pulmonary application, certain adjuvants for solubilization which may be used in the case of oral administration cannot be employed for lack of tolerability. For example, Sandimmun® Optoral capsules (Novartis) which contain cyclosporine A, comprise a microemulsion concentrate with ethanol, propylene glycol and significant amounts of surfactants and, therefore, constitute a formulation which, if inhaled, would cause serious toxic effects. Similarly, the Sandimmun® infusion solution concentrate (Novartis), which is available for infusion, is also not inhalable: The only adjuvants contained therein are ethanol and poly(oxyethylene)-40-castor oil. It can be used for infusion only because it is previously diluted with a 0.9% sodium chloride solution or a 5% glucose solution, at a ratio of 1:20 to 1:100. This results in large volumes which can be administered by infusion, but not by inhalation.

WO 86/03938 describes a method for preserving liposomes containing biologically active molecules, such that when rehydrated, liposome structures retain substantially all material originally encapsulated. A preserving agent having at least two monosaccharide units, such as trehalose is used either internally or externally or both.

WO 90/00389 describes a freeze-dried potential liposome mixture having an amphipathic lipid and a cyclosporin or derivative thereof for use in possible liposome delivery of cyclosporin into cells. The freeze-dried potential liposome mixture can include various saccharides such as sucrose, trehalose, and glucose to impart cryoprotective effects and enhance long-term stability of the liposomes. When reconstituted to yield liposomes in an aqueous medium, substantially all of the cyclosporin present in the freeze-dried mixture is encapsulated in the liposomes. The document further discloses a freeze-dried liposome mixture comprising an amphipathic lipid and a cyclosporin or derivative thereof, said mixture being essentially free of saccharide and being storable for at least 120 days and wherein at least 90% of said cyclosporin in said mixture is encapsulated in liposomes of substantially uniform particle size distribution when an aqueous liposome preparation is prepared subsequent to said storage.

WO 92/18104 discloses a process of preparing a liposomal cyclosporine therapeutic formulation, which is characterized by dissolving a neutral phospholipid, a negatively charged phospholipid selected from phosphatidylglycerols and dimyristoylphosphatidic acid, and a cyclosporine in an organic solvent to form a solution; drying the solution to form a solid phase; hydrating the solid phase with an aqueous solution to form the liposomal cyclosporine therapeutic formulation. The document further describes liposomes suspended in an aqueous solution and characterized by comprising a neutral phospholipid, a negatively charged phospholipid selected from phosphatidylglycerols and dimyristoylphosphatidic acid, and a cyclosporine.

WO 96/40064 discloses liposomal encapsulated cyclosporin formulations which are efficacious as immunosuppressant agents and in the treatment of drug resistant cancers. The formulations include liposomes comprised of a phosphatidylcholine, cholesterol, a phosphatidylglycerol and a cyclosporin, e.g. of phosphatidylcholine, cholesterol, dimyristoylphosphatidylglycerol and cyclosporin. The liposomes are described to be unilamellar and to have a size of less than 75 nanometers, and to be stable in whole mammal blood.

WO 98/00111 proposes a liposomal dispersion of cyclosporine A for inhalation having a very high concentration of phospholipid of up to 225 mg/ml. However, this has such a high dynamic viscosity that it cannot be nebulized efficiently. A liposomal preparation of cyclosporine A is also known from US 2003/0215494. The invention described therein, however, lies in the fact that such a preparation is to be used for the inhibition of pulmonary metastases. U.S. Pat. No. 5,958,378 describes liposomal cyclosporine preparations for nebulization; however, the viscosity thereof is so high that these cannot be nebulized with an electronic vibrating membrane nebulizer. Moreover, the organic solvent butanol is used for the preparation thereof, but despite a subsequent lyophilization process, this cannot be removed completely and yields liposomes of >1 μm, which cannot be sterilized by filtration and which have only a low ability to permeate epithelial cell membranes.

WO 98/36736 describes a lyophilized composition comprising trehalose and lipid liposomes in which a biologically active principle has been incorporated, characterized in that the biologically active principle is highly insoluble in water, the trehalose/lipid weight ratio is smaller or equal to 1.5, and all of the trehalose was added to the outside of the liposomes already formed before lyophilization.

WO 03/099362 A1 describes a method of inhibiting growth of lung metastases in an individual comprising the steps of administering a dose of a lipid-drug enhancer liposomal complex; and, in sequence, administering a dose of a lipid-anticancer drug liposomal complex, both of said liposomal complexes delivered via aerosolization from a nebulizer; whereby said drug enhancer and said anticancer drug inhibit growth of lung metastases in the individual, wherein said drug enhancer may be selected from cyclosporin A, cyclosporin D, verapamil, ketoconazole, PCS 833, erythromycin, nifedipine, rapamycin or 20 mibefradil and wherein said anti-cancer drug may be selected from paclitaxel, doxorubicin, etoposide, vinblastine, camptothecins, cisplatinum, carboplatinum, daunorubicin, or adriamycin.

WO 2007/065588 A1 discloses liquid pharmaceutical compositions containing a therapeutically effective dose of a cyclosporin; an aqueous carrier liquid; a first solubilizing substance selected among the group of phospholipids; and a second solubilizing substance selected among the group of non-ionic surfactants. The disclosed composition is suitable for oral, parenteral, nasal, mucosal, topical, and particularly pulmonary application in the form of an aerosol.

WO 2016/146645A1 discloses liposomal cyclosporine formulations that preferably comprise unilamellar liposomes. The liposomes preferably have an average diameter of at most about 100 nm measured as z-average using photon correlation spectroscopy and a polydispersity index of at most about 0.5 as measured by photon correlation spectroscopy.

The formulation can be presented as a solid formulation for reconstitution with an aqueous solvent immediately before inhalation. The solid formulation can be prepared by any method suitable for removing the solvent from a liquid formulation. Preferred examples of methods for preparing such solid formulation are freeze drying and spray drying. To protect the active ingredient during the drying process, it may be useful to incorporate lyoprotective and/or bulking agents, such as a sugar or a sugar alcohol, in particular sucrose, fructose, glucose, trehalose, mannitol, sorbitol, isomalt, or xylitol. Most notably, however, the sugar is added to the preformed formulation comprising the liposomal encapsulated CsA.

Thus, it is an object of the present invention to provide an improved pharmaceutical formulation comprising CsA in liposomally solubilized form which allows for the pulmonary administration of CsA, preferably by inhalation, which is easy to prepare from readily available starting materials and with readily available techniques. Furthermore, the pharmaceutical formulation comprising CsA in liposomally solubilized form should allow for enhanced stability of the formulation or, more specifically the liposomes comprised by the formulation compared to liquid formulations to allow for longer storage periods and reduced sensitivity to temperature or other storage conditions. Furthermore, the improved pharmaceutical formulation comprising CsA in liposomally solubilized form, especially when provided as a solid, should allow for an easy and fast reconstitution and retention of the physical characteristics of the liposomes comprised. For objects of the present invention will become apparent from the present disclosure including the examples and claims.

### SUMMARY OF THE INVENTION

In the first aspect, the invention relates to a lyophilized pharmaceutical composition for reconstitution in an aqueous carrier liquid comprising:
a) liposome-forming structures comprising
   i. a therapeutically effective amount of cyclosporine A (CsA);
   ii. a membrane-forming substance selected from the group of phospholipids; and
   iii. a solubility-enhancing substance selected from the group of non-ionic surfactants;
and
b) at least one disaccharide selected from the group consisting of saccharose, lactose and trehalose,
   wherein the at least one disaccharide is present in an amount of at least 40 wt.-% with regard to the total weight of the lyophilized composition.

In a second aspect, the present invention relates to a lyophilized pharmaceutical composition of the first aspect of the invention for use as a medicament for pulmonary application.

In a third aspect, the present invention relates to a kit for the preparation of an aqueous liposomal dispersion for inhalation comprising a therapeutically effective amount of cyclosporine A in liposomally solubilized (incorporated/intercalated) form, comprising
a lyophilized pharmaceutical composition according to the first aspect of the invention, and
an aqueous carrier liquid.

In a fourth aspect, the present invention relates to a process for the preparation of an aqueous liposomal dispersion for inhalation comprising cyclosporine A in liposomally solubilized form by reconstitution of the lyophilized pharmaceutical composition of the first aspect of the invention, comprising dispersing the lyophilized pharmaceutical composition according to the first aspect of the invention in an aqueous carrier liquid.

In a fifth aspect, the present invention relates to a liquid liposomal dispersion comprising an aqueous carrier liquid and a therapeutically effective amount of cyclosporine A in liposomally solubilized form, prepared by a process comprising dispersing the lyophilized pharmaceutical composition according to the first aspect of the invention in an aqueous carrier liquid.

In a sixth aspect, the present invention provides a process for the preparation of a lyophilized pharmaceutical composition comprising a therapeutically effective amount of cyclosporine A in liposomally solubilized form for reconstitution in an aqueous carrier liquid, the process comprising the steps:
(a) providing a liquid aqueous dispersion of liposomes, said liposomes comprising:
   i. a therapeutically effective amount of cyclosporine A (CsA);
   ii. a membrane-forming substance selected from the group of phospholipids; and
   iii. a solubility-enhancing substance selected from the group of non-ionic surfactants; and optionally
   iv. one or more further excipients, such as buffers and/or chelating agents;
wherein said liquid aqueous dispersion further comprises at least one disaccharide selected from the group consisting of saccharose, lactose and trehalose, which is present in an amount of at least 40 wt.-% with regard to the total weight of the lyophilized composition; and
(b) lyophilizing said aqueous dispersion.

In a seventh aspect, the present invention relates to a lyophilized pharmaceutical composition obtainable by a process comprising the steps of
(a) providing a liquid aqueous dispersion of liposomes, said liposomes comprising:
   i. a therapeutically effective amount of cyclosporine A (CsA);
   ii. a membrane-forming substance selected from the group of phospholipids; and
   iii. a solubility-enhancing substance selected from the group of non-ionic surfactants; and optionally
   iv. one or more further excipients, such as buffers and/or chelating agents;
wherein said liquid aqueous dispersion further comprises at least one disaccharide selected from the group consisting of saccharose, lactose and trehalose, which is present in an amount of at least 40 wt.-% with regard to the total weight of the lyophilized composition; and
(b) lyophilizing said aqueous dispersion.

In a specific embodiment, the seventh aspect of the present invention relates to a lyophilized pharmaceutical composition obtained or obtainable by a process according to the fourth aspect of the present invention.

### DETAILED DESCRIPTION OF THE INVENTION

The terms “consist of”, “consists of” and “consisting of” as used herein are so-called closed language meaning that only the mentioned components are present. The terms “comprise”, “comprises” and “comprising” as used herein are so-called open language, meaning that one or more further components may or may not also be present.

The term “active pharmaceutical ingredient” (also referred to as “API” throughout this document) refers to any type of pharmaceutically active compound or derivative that is useful in the prevention, diagnosis, stabilization, treatment, or—generally speaking—management of a condition, disorder or disease.

The term “therapeutically effective amount” as used herein refers to a dose, concentration or strength which is useful for producing a desired pharmacological effect. In the context of the present invention, the term “therapeutically effective” also includes prophylactic activity. The therapeutic dose is to be defined depending on the individual case of application. Depending on the nature and severity of the disease, route of application as well as height and state of the patient, a therapeutic dose is to be determined in a way known to the skilled person.

In the context of the present invention, a “pharmaceutical composition” is a preparation of at least one API and at least one adjuvant, which, in the simplest case, can be, for example, an aqueous liquid carrier such as water or saline.

‘A’ or ‘an’ does not exclude a plurality; i.e. the singular forms ‘a’, ‘an’ and ‘the’ should be understood as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. The terms ‘a’, ‘an’ and ‘the’ hence have the same meaning as ‘at least one’ or as ‘one or more’ unless defined otherwise. For example, reference to ‘an ingredient’ includes mixtures of ingredients, and the like.

When used herein the term ‘about’ or ‘ca.’ will compensate for variability allowed for in the pharmaceutical industry and inherent in pharmaceutical products, such as differences in content due to manufacturing variation and/or time-induced product degradation. The term allows for any variation, which in the practice of pharmaceuticals would allow the product being evaluated to be considered bioequivalent in a mammal to the recited strength of a claimed product.

‘Essentially’, ‘about’, ‘approximately’, ‘substantially” and the like in connection with an attribute or value include the exact attribute or the precise value, as well as any attribute or value typically considered to fall within a normal range or variability accepted in the technical field concerned. For example, ‘substantially free of water” means that no water is deliberately included in a formulation, but does not exclude the presence of residual moisture.

In the context of the present invention, a “colloidal aqueous solution” preferably means a solution without organic solvent consisting of mainly unilamellar liposomes having a mean diameter of at most 100 nm and/or a polydispersity index (PI) of not more than 0.50 in which the active agent is, at least predominantly, dissolved. Preferably, water, or more specifically saline is the only liquid solvent contained in the preparation. Furthermore, it is preferred that the preparation is an aqueous solution or an aqueous colloidal solution, i.e., a monophasic liquid system. Such a system is essentially free of dispersed particles having a greater than colloidal particle size. By convention, particles below about 1 μm are regarded as colloidal particles which do not constitute a separate phase and do not result in a physical phase boundary. Sometimes, particles in a size range just above 1 μm are also still considered colloidal. Preferably, however, colloidal aqueous solutions as used herein are essentially free of particles which do clearly not belong to the colloidal spectrum, i.e., for example, particles having a diameter of 1 μm or more.

According to the first aspect, the present invention provides a lyophilized pharmaceutical composition for reconstitution in an aqueous carrier liquid comprising:
a) liposome-forming structures comprising
   i. a therapeutically effective amount of cyclosporine A (CsA);
   ii. a membrane-forming substance selected from the group of phospholipids; and
   iii. a solubility-enhancing substance selected from the group of non-ionic surfactants;
and
b) at least one disaccharide selected from the group consisting of saccharose, lactose and trehalose,
   wherein the at least one disaccharide is present in an amount of at least 40 wt.-% with regard to the total weight of the lyophilized composition.

The pharmaceutical composition according to the present invention may be prepared by lyophilization of a precursor solution or preferably dispersion as described in further detail below. The term “lyophilization” or “lyophilized” as used herein means a process by which an aqueous or non-aqueous solution or dispersion comprising dissolved or dispersed components are dried under reduced pressure and usually at low temperatures by sublimation as described in further detail below. In preferred embodiments, the pharmaceutical compositions of the present invention may be prepared by removal of an aqueous solvent, carrier liquid, liquid vehicle or continuous phase of a precursor solution or dispersion.

The lyophilized pharmaceutical compositions of the present invention are suitable for and may be reconstituted in an aqueous carrier liquid. The term “reconstituted” as used herein means that the lyophilized pharmaceutical compositions obtained or generated by the lyophilization process in form of a solid material may be re-dissolved or re-dispersed, preferably re-dispersed in an aqueous carrier liquid.

The lyophilized pharmaceutical compositions according to the present invention comprise liposome-forming structures as a component a). Said liposome-forming structures comprise cyclosporine A (CsA) in a therapeutically effective amount as a first constituent, a membrane-forming substance selected from the group of phospholipids as a second component, and a solubility-enhancing substance selected from the group of non-ionic surfactants as third component. In specific embodiments, the lyophilized pharmaceutical compositions according to the present invention may optionally further comprise one or more (further) excipients, for example buffering agents or chelating agents as described in further detail below.

The lyophilized pharmaceutical compositions of the present invention further comprise as a component b) at least one disaccharide selected from the group consisting of saccharose (sucrose; the terms ‘saccharose’ and ‘sucrose’ as used herein have the same meaning and are used synonymously for β-D-Fructofuranosyl α-D-glucopyranoside; CAS number 57-50-1), lactose (β-D-Galactopyranosyl-(1→4)-D-glucose; CAS number 63-42-3) and trehalose (α-D-glucopyranosyl-(1→1)-α-D-glucopyranoside; CAS number 99-20-7), wherein the at least one disaccharide is present in an amount of at least about 40 wt.-% with regard to the total weight of the lyophilized composition. In some embodiments, the at least one disaccharide is present in an amount of from at least about 40 wt.-% up to about 95 wt.-% or up to about 90 wt.-% or up to about 85 wt.-% or up to about 80 wt.-%, all with regard to the total weight of the lyophilized composition. In specific embodiments, the pharmaceutical composition of the present invention comprises saccharose and/or trehalose, preferably saccharose as the disaccharide which is present in an amount of at least about 40 wt.-% with regard to the total weight of the lyophilized composition. In further embodiments, the pharmaceutical composition of the present invention comprises trehalose as the disaccharide which is present in an amount of at least about 40 wt.-% with regard to the total weight of the lyophilized composition. In specific embodiments, the pharmaceutical compositions of the present invention may comprise mixtures of all three disaccharides, namely saccharose, lactose and trehalose or mixtures of two of the named disaccharides, namely mixtures of saccharose and lactose or mixtures of saccharose an trehalose or mixtures of lactose and trehalose. In these cases, the amount of at least 40 wt-% with regard to the total weight of the lyophilized composition refers to the combined weight of the respective disaccharides comprise by the lyophilized composition according to the present invention.

The liposome-forming structures of component a) of the pharmaceutical compositions of the present invention comprise mainly lipid bilayer membranes formed of the membrane forming substance or substances selected from the group of phospholipids according to item ii. The liposome-forming structures as used herein are capable of forming liposomes, preferably liposomes with a closed, spherical bilayer membrane and an inner lumen upon contact or reconstitution with an aqueous carrier liquid as described in further detail below.

The liposome-forming structures according to component a) of the present lyophilized pharmaceutical compositions comprise a bilayer membrane formed of the membrane-forming substance selected from the group of phospholipids. The liposome-forming structures as used herein, however, may or may not have a continuous or closed bilayer membrane. In specific embodiments, the liposome-forming structures may at least be partly present in unilamellar form or, preferably, may predominantly be present in unilamellar form. The term “unilamellar” as used herein means that the corresponding liposome-forming structures only comprise a single layer formed by a single lipid bilayer membrane and not a plurality of lipid bilayer membranes in a layered arrangement.

In specific embodiments, the liposome-forming structures of component a) of the present lyophilized pharmaceutical composition may comprise an inner lumen surrounded or at least partially surrounded by the bilayer membrane formed of the membrane forming substance selected from the group of phospholipids. In further specific embodiments, however, the liposome-forming structures may have the shape of collapsed or partly collapsed spheres or spheroids built by a lipid bilayer membrane or by a plurality of lipid bilayer membranes attached to or associated with each other with or without openings between the inner lumen and the surrounding.

The inner lumen of the liposome-forming structures of the present lyophilized pharmaceutical compositions may or may not contain residual water or aqueous carrier liquid. In preferred embodiments, however, the inner lumen of the liposome-forming structures is at least partially dehydrated. Accordingly, in specific embodiments, the inner lumen of the liposome-forming structures as well as the liposome- forming structures in general contain or comprise only residual amounts of water or of the aqueous carrier liquid. Furthermore, it should be noted that the inner lumen of the liposome-forming structures may or may not contain or comprise (fractions of) the at least one disaccharide selected from the group consisting of saccharose, lactose and trehalose, preferably trehalose and/or saccharose.

In preferred embodiments, the lyophilized pharmaceutical compositions according to the present invention comprise the at least one disaccharide, preferably saccharose and/or lactose, especially saccharose, in an amount selected in the range of from about 50 wt.-% to about 80 wt.-% or to about 75 wt.-%, with regard to or based on the total weight of the lyophilized composition. In further preferred embodiments, the lyophilized pharmaceutical compositions according to the present invention comprise the at least one disaccharide, preferably saccharose and/or lactose, especially saccharose in an amount selected in the range of from about 60 wt.-% to about 75 wt.%, even more preferably selected in the range of from about 65 wt.-% to about 70 wt.-% with regard to the total weight of the lyophilized composition.

The present lyophilized pharmaceutical compositions comprise a therapeutically effective amount of cyclosporine A (CsA), wherein cyclosporine A is defined chemically as cyclo-[[(E)-(2S,3R,4R)-3-hydroxy-4-methyl-2-(methylamino)-6-octenoyl]-L-2-aminobutyryl-N-methylglycyl-N-methyl-L-leucyl-L-valyl-N-methyl-L-leucyl-L-alanyl-D-alanyl-N-methyl-L-leucyl-N-methyl-L-leucyl-N-methyl-L-valyl] and is a cyclic peptide with immunosuppressive activity.

In specific embodiments, the therapeutically effective amount of CsA as comprised by the liposome-forming structures according to the present invention is at least partially incorporated (or intercalated) in the bilayer membrane of the liposome-forming structures. The term “incorporated” as used herein means, with regard to CsA being a lipophilic compound, that CsA is located or intercalated in the inner lipophilic part of the bilayer lipid membrane rather than on the hydrophilic outer surfaces of the lipid bilayer membrane (whereas the terms surfaces can mean both surfaces, or more specifically the inner or outer surface of the bilayer membrane forming the liposome-forming structures).

In preferred embodiments, the CsA is predominantly incorporated in the bilayer membrane of the liposome-forming structures. In exemplary embodiments, at least about 90% or even at least about 95% or even at least about 97.5% of the total amount of CsA comprised by the present lyophilized pharmaceutical composition is incorporated in the bilayer membranes of the liposome-forming structures of the present invention. In further exemplary embodiments, about 90% or about 95% to about 97.5% or to about 99% or 99.5% or even 99.9% of the total amount of CsA comprised by the present lyophilized pharmaceutical composition is incorporated in the bilayer membranes of the liposome-forming structures of the present invention.

In further preferred embodiments, the present lyophilized pharmaceutical compositions comprise CsA in an amount in the range of from about 2 to about 4 wt.-%, preferably of from about 2.2 to about 3.4 wt.-% or even more preferably of from about 2.4 to about 3.4 wt.-% or from about 2.4 wt.-% to about 3.0 wt.-%, or from about 2.5 wt.-% to about 2.9 wt.-% or from about 2.6 wt.-% to about 2.8 wt.-% or from about 2.65 wt.-% to about 2.75 wt.-%, in each case based on the weight of the lyophilized composition. In further specific embodiments of the present lyophilized pharmaceutical compositions, the ratio of the weight of the at least one disaccharide according to component b) to the weight of cyclosporine A in the lyophilized composition is selected in the range of from about 10:1 to about 30:1, or from about 20:1 to about 30:1 or from about 20:1 to about 27.5:1 or even from about 22.5:1 to about 27.5:1.

The liposome-forming structures according to component a) of the lyophilized pharmaceutical compositions of the present invention further comprise as an item ii.

a membrane-forming substance selected from the group of phospholipids or a mixture of two or more different membrane-forming substances selected from the group of phospholipids. The term “membrane-forming substance” as used herein means that the substance is capable of forming a lipid bilayer membrane by self-assembly in an aqueous carrier liquid, such as water or saline and/or is capable of forming liposomes in an aqueous carrier liquid under circumstances as described in further detail below.

Preferred phospholipids comprised by the liposome forming structures of the present invention are, in particular, mixtures of natural or enriched phospholipids, for example, lecithines such as the commercially available Phospholipon® G90, 100, or Lipoid 90, S 100. Accordingly, in preferred embodiments, the membrane-forming substance selected from the group of phospholipids is a mixture of natural phospholipids.

Phospholipids are amphiphilic lipids which contain phosphorus. Known also as phosphatides, they play an important role in nature, especially as the double layer forming constituents of biological membranes and frequently used for pharmaceutical purposes are those phospholipids which are chemically derived from phosphatidic acid. The latter is a (usually doubly) acylated glycerol-3-phosphate in which the fatty acid residues may be of different lengths. The derivatives of phosphatidic acids are, for example, the phosphocholines or phosphatidylcholines, in which the phosphate group is additionally esterified with choline, as well as phosphatidylethanolamine, phosphatidylinositols etc. Lecithins are natural mixtures of various phospholipids which usually contain a high proportion of phosphatidylcholines. Preferred phospholipids according to the invention are lecithins as well as pure or enriched phosphatidylcholines such as dimyristoylphospatidylcholine, di-palmitoyl-phosphatidylcholine and distearoylphosphatidylcholine.

In further preferred embodiments, the membrane-forming substance selected from the group of phospholipids is a lecithin containing unsaturated fatty acid residues. In yet further preferred embodiments, the membrane-forming substance selected from the group of phospholipids is a lecithin selected from the group consisting of soy bean lecithin, Lipoid S100, Phospholipon® G90, 100 or a comparable lecithin. In further preferred embodiments, the membrane-forming substance selected from the group of phospholipids is selected from Lipoid S100, Lipoid S75, particularly Lipoid S100.

In specific embodiments, the content of the membrane-forming substance selected from the group of phospholipids, preferably Lipoid S100, is from about 10 or 15 wt.-% to about 30 wt.-% and preferably from about 20 to about 30 wt.-%, and even more preferably from about 23 to about 27 wt.-% based on the total weight of the lyophilized composition. In further specific embodiments, the weight ratio of the membrane forming substance selected from the group of phospholipids as described above to CsA is selected in the range of from about 8:1 to about 11:1, preferably from about 8.5:1 to about 10:1, for example, about 9:1.

The liposome-forming structures according to component a) of the lyophilized pharmaceutical compositions of the present invention further comprise as an item iii. a solubility-enhancing substance or a mixture of two or more different solubility-enhancing substances selected from the group of non-ionic surfactants. Non-ionic surfactants have—as other surfactants—at least one rather hydrophilic and at least one rather lipophilic molecular region. There are monomeric, low molecular weight non-ionic surfactants and non-ionic surfactants having an oligomeric or polymeric structure. Examples of suitable non-ionic surfactants suitable as solubility-enhancing substances according to item iii. of the liposome-forming structures of the present invention comprise polyoxyethylene alkyl ethers, polyoxyethylene sorbitan fatty acid esters such as, for example, polyoxyethylene sorbitan oleate, sorbitan fatty acid esters, poloxamers, vitamin E-TPGS (D-a-tocopheryl polyethylene glycol 1000 succinate) and tyloxapol.

In specific embodiments, the solubility-enhancing substance selected from the group of non-ionic surfactants selected from the group of polysorbates and vitamin E-TPGS, preferably is selected from the group of polysorbates. In a particularly preferred embodiment, the solubility-enhancing substance selected from the group of non-ionic surfactants is polysorbate 80.

The content of the solubility-enhancing substance selected from the group of non-ionic surfactants may preferably be chosen in the range of from about 0.01 to about 5 wt.-%, or from about 0.1 to about 4 wt.-%, or from about 0.5 to about 3.5 wt.-%, or from about 1 to about 3 wt.-%, preferably from about 1.5 to about 2.5 wt.-%, or from about 1.6 wt.-% to about 2.3 wt-%, or from 1.7 wt.-% to about 2.1 wt.% or from about 1.8 to about 2.0 wt.%, in each case based on the total weight of the lyophilized composition.

In specific embodiments of the present lyophilized pharmaceutical compositions, the amount of the membrane-forming substance selected from the group of phospholipids, preferably the lecithin is larger than the amount of the solubility-enhancing substance selected from the group of non-ionic surfactants. In exemplary embodiments, the weight ratio of the membrane forming substance selected from the group of phospholipids, preferably the lecithin, to the solubility enhancing substance selected from the group of non-ionic surfactants, preferably the polysorbate, is selected in the range of from about 15:1 to about 9:1, preferably from about 14:1 to about 12:1, for example, about 13:1.

In further specific embodiments, the weight ratio between the (sum of the) membrane-forming substance selected from the group of phospholipids and the solubility-enhancing substance selected from the group of non-ionic surfactant on the one hand and CsA on the other hand is selected in the range of from about 5:1 to about 20:1, preferably from about 8:1 to about 12:1 and more preferably about 10:1.

In yet further specific embodiments, the weight ratio between the membrane-forming substance selected from the group of phospholipids, preferably the lecithin, the solubility-enhancing substance selected from the group of non-ionic surfactants, preferably the polysorbate and CsA is selected in the range of from about 15:1:1.5 to about 5:0.3:0.5, and preferably at about 9:0.7:1.

The lyophilized pharmaceutical composition according to the present invention may or may not further comprise residual water after lyophilization, which may be associated to the surfaces of the liposome-forming structures or which may be contained in the inner lumen of the liposome-forming structures as described above. In preferred embodiments, the amount of residual water comprised by the lyophilized composition is in the range of up to about 5 wt.-%, or up to about 3 wt.-%, or preferably up to about 2 wt.-%, based on the total weight of the lyophilized pharmaceutical composition.

The lyophilized pharmaceutical composition of the present invention may further comprise one or more further excipients as an optional component c). Suitable excipients are known to the skilled person. For example, the lyophilized pharmaceutical composition can optionally contain pH-correcting agents in order to adjust the pH, such as physiologically acceptable bases, acids or salts, optionally as buffer mixtures. In this context, the term “physiologically acceptable” does not mean that one of the excipients must be tolerable on its own and in undiluted form, which would not be the case, for example, for sodium hydroxide solution, but means that it must be tolerable at the concentration in which it is contained in the lyophilized pharmaceutical composition, especially after reconstitution.

Suitable pH-correcting agents or buffers for adjusting the pH may be selected, inter alfa, with regard to the intended route of application. Examples for potentially useful excipients of this group comprise sodium hydroxide solution, basic salts of sodium, calcium or magnesium such as, for example, citrates, phosphates, acetates, tartrates, lactates etc., amino acids, acidic salts such as hydrogen phosphates or dihydrogen phosphates, especially those of sodium, moreover, organic and inorganic acids such as, for example, hydrochloric acid, sulphuric acid, phosphoric acid, citric acid, cromoglycinic acid, acetic acid, lactic acid, tartaric acid, succinic acid, fumaric acid, lysine, methionine, acidic hydrogen phosphates of sodium or potassium etc.

In one of the advantageous embodiments of the invention, the lyophilized pharmaceutical composition comprises buffers to ensure a neutral or acidic pH of the pharmaceutical composition after reconstitution. Preferably, the pH of the present pharmaceutical composition after reconstitution is in the range of at most about 8.5 or in the range of about 2.5 to about 7.5. For pulmonary or parenteral application, a pH within the range of from about 4 to about 7.5 is preferred, provided that this is compatible with other requirements of the formulation such as, for example, stability aspects. Particularly preferred is a composition which is buffered with a phosphate buffer to ensure a pH in the range of 6.0 to 7.5 or from 6.0 to 7.0 or in the range of from 6.3 to 6.7 after reconstitution, whereby the stability of the composition can be markedly improved and the occurrence of undesirable lysolecithin during storage can be effectively reduced.

Furthermore, the present lyophilized pharmaceutical composition may or may not contain osmotically active adjuvants in order to adjust it to a desired osmolality after reconstitution, which is important in certain applications such as especially for for inhalation, in order to achieve good tolerability. Such adjuvants are frequently referred to as ‘isotonizing agents’ even if their addition does not necessarily result in an isotonic composition after reconstitution, but in an isotonicity close to physiological osmolality in order to achieve the best possible physiological tolerability.

A particularly frequently used isotonizing agent is sodium chloride, but this is not suitable in every case. In an advantageous embodiment of the invention, the preparation contains no sodium chloride, except, of course, natural ubiquitous sodium chloride amounts which may also be contained in water of pharmaceutical quality. In another embodiment, the lyophilized pharmaceutical composition contains an essentially neutral salt as isotonizing agent which is not sodium chloride, but, for example, a sodium sulphate or sodium phosphate. It should be noted, however, that the isotonizing agent may also be comprised by the aqueous carrier liquid, for example in form of an aqueous solution of sodium chloride (saline). In this case, however, salts other than sodium salts may be also preferable. Thus, it is known that certain calcium and magnesium salts have a positive or supporting effect in the inhalation of active agent solutions, possibly because they themselves counteract the local irritations caused by the administration and because they have a bronchodilatory effect which is currently postulated in the clinical literature (for example Hughes et al., Lancet. 2003; 361 (9375): 2114-7) and/or because they inhibit the adhesion of germs to the proteoglycans of the mucosa of the respiratory tract so that the mucociliary clearance as the organism's natural defense against pathogens is supported indirectly (K. W. Tsang et al., Eur. Resp. 2003. 21, 932-938). Advantageous may be, for example, magnesium sulphate, which has excellent pulmonary tolerability and can be inhaled without concern, as well as calcium chloride (1-10 mmol).

In further specific embodiments, the lyophilized pharmaceutical compositions according to the present invention comprise one or more further excipients which are selected from buffers and chelating agents. Exemplary compounds suitable as buffers for the adjustment of the pH of the present pharmaceutical compositions after reconstitution comprise, for example, sodium dihydrogen phosphate dihydrate and/or disodium hydrogen phosphate dodecahydrate, sodium hydroxide solution, basic salts of sodium, calcium or magnesium such as, for example, citrates, phosphates, acetates, tartrates, lactates etc., amino acids, acidic salts such as hydrogen phosphates or dihydrogen phosphates, especially those of sodium, moreover, organic and inorganic acids such as, for example, hydrochloric acid, sulphuric acid, phosphoric acid, citric acid, cromoglycinic acid, acetic acid, lactic acid, tartaric acid, succinic acid, fumaric acid, lysine, methionine, acidic hydrogen phosphates of sodium or potassium etc. and further buffer systems as described above. In further specific embodiments, the lyophilized pharmaceutical compositions according to the present invention comprise one or more further excipient which are selected from chelating agents, for example, disodium edetate dihydrate, calcium sodium EDTA, preferably disodium edetate dihydrate.

In further embodiments, the lyophilized composition of the present invention may essentially consist of CsA in a pharmaceutically effective amount; the membrane-forming substance or substances selected from the group of phospholipids; the solubility-enhancing substance or substances selected from the group of non-ionic surfactants; the at least one disaccharide selected from the group consisting of saccharose, lactose and trehalose, wherein the at least one disaccharide is present in an amount of at least 40 wt.-% with regard to the total weight of the lyophilized composition; and optionally further excipients and optionally residual amounts of water.

In an exemplary embodiment, the present lyophilized pharmaceutical composition comprises, or, together with further optional excipients, essentially consist or consist of, preferably comprises (each based on the total weight of the lyophilized pharmaceutical composition):
Cyclosporine A: 2-4 wt.-%
Disaccharide: 40-80 wt.-%
Phospholipid: 10-40 wt.-%
Non-ionic surfactant: 0.01-10 wt.-%
Buffering agent: 1-6 wt.-%
Chelating agent: 0.05-0.5 wt.-%,
   wherein the sum of the components add to 100 wt.-% of the final lyophilized pharmaceutical composition. It should be noted that that the values and ranges given above are calculated on the basis of a lyophilized and completely anhydrous composition. For practical reasons, however, the lyophilized composition in addition to the components listed above may or may not contain residual amounts of water in the range of from about 0 to about 5 wt.-% based on the weight of the lyophilized pharmaceutical composition.

In a preferred exemplary embodiment, the present lyophilized pharmaceutical composition comprises, or, together with further optional excipients, essentially consist or consist of, preferably comprises (each based on the total weight of the lyophilized pharmaceutical composition):
Cyclosporine A: 2-4 wt.-%
Disaccharide: 50-75 wt.%
Phospholipid: 15-40 wt.-%
Non-ionic surfactant: 0.1-4 wt.-%
Buffering agent: 2-6 wt.-%
Chelating agent: 0.05-0.5 wt.-%,
   wherein the sum of the components add to 100 wt.-% of the final lyophilized pharmaceutical composition and wherein the lyophilized composition in addition to the components listed above may or may not contain residual amounts of water in the range of from about 0 to about 2 wt.-% based on the weight of the lyophilized pharmaceutical composition.

In a particularly preferred exemplary embodiment, the present lyophilized pharmaceutical composition comprises, or, together with further optional excipients, essentially consist or consist of, preferably comprises (each based on the total weight of the lyophilized pharmaceutical composition):
Cyclosporine A: 2.5-3 wt.-%
Disaccharide: 60-75 wt. %
Phospholipid: 20-30 wt.-%
Non-ionic surfactant: 1-3 wt.-%
Buffering agent: 3-5 wt.-%
Chelating agent: 0.05-0.2 wt.-%,
   wherein the sum of the components add to 100 wt.-% of the final lyophilized pharmaceutical composition and wherein the lyophilized composition in addition to the components listed above may or may not contain residual amounts of water in the range of from about 0 to about 2 wt.-% based on the weight of the lyophilized pharmaceutical composition. In these compositions the “Phospholipid” preferably is Lipoid S75 or Lipoid S100, preferably Lipoid S100; the “Disaccharide” preferably is saccharose (sucrose); and the non-ionic surfactant” preferably is a polysorbate, especially polysorbate 80.

As described above, the lyophilized pharmaceutical compositions may be dissolved or dispersed, preferably dispersed in an aqueous carrier liquid such as water or saline, preferably in sterilized form to yield colloidal aqueous dispersions of CsA in liposomally solubilized form (herein also referred to as ‘L-CsA’) as described in further detail below. One of the key advantages of the lyophilized pharmaceutical compositions according to the present invention is the enhanced and prolonged stability of the liposomally solubilized CsA compared to other pharmaceutical compositions comprising CsA in liposomally encapsulated form. Furthermore, the lyophilized pharmaceutical compositions according to the present invention may be easily and readily reconstituted by dispersion in an aqueous carrier liquid, wherein the liposomes formed by reconstitution of the liposome-forming structures show an average size distribution comparable to that of the liposomes initially formed prior to lyophilization as described in further detail below.

Without wishing to be bound by theory, this may be attributed to the stabilizing effect of the disaccharide selected from the group of saccharose, lactose and trehalose and present in an amount of at least 40 wt.-%, based on the total weight of the lyophilized pharmaceutical composition. Furthermore, the above-described beneficial properties of the lyophilized pharmaceutical compositions of the present invention may be attributed to the fact that the disaccharide selected from the group consisting of saccharose, lactose and trehalose, preferably saccharose, is present on the outside as well in the inner lumen of the liposome-forming structures.

The pharmaceutical compositions of the present invention may be prepared as described in detail below and, after lyophilization, usually are obtained in the form of colorless or almost colorless solids or powders.

In a second aspect, the present invention provides a lyophilized pharmaceutical composition for reconstitution in an aqueous carrier liquid comprising:
a) liposome-forming structures comprising
   i. a therapeutically effective amount of cyclosporine A (CsA);
   ii. a membrane-forming substance selected from the group of phospholipids; and
   iii. a solubility-enhancing substance selected from the group of non-ionic surfactants; and
b) at least one disaccharide selected from the group consisting of saccharose, lactose and trehalose, wherein the at least one disaccharide is present in an amount of at least 40 wt.-% with regard to the total weight of the lyophilized composition, for use as a medicament, especially for pulmonary application.

As described above, the compositions of the present invention can be used as medicaments, especially after reconstitution in an aqueous carrier liquid, for example for the prophylaxis and treatment of autoimmune diseases, skin diseases, after transplantations or diseases of the sensory organs (eyes, nose, ear), malaise and pulmonary diseases, for example, asthma, chronic obstructive bronchitis, parenchymal, fibrotic and interstitial lung diseases or inflammations, lung cancer, and preferably for the prevention and treatment of acute or chronic transplant rejection reactions and the diseases resulting therefrom such as bronchiolitis obliterans, especially after lung, heart, bone marrow or stem cell transplantations, especially preferred after lung transplantations. It may further be used to increase the efficacy of other medicaments, in particular, of cytostatics, where an additive or synergistic effect may be achieved with cyclosporine through the efflux pump inhibition effect. Nasal, oral, ophthalmic, mucosal, parenteral or topical application of the composition according to the present invention can, in individual cases, be advantageous. The administration may be affected by application, dropping, spraying onto or into the body, which, in initial tests on humans, proved to be particularly well tolerated.

Preferably, however, the lyophilized pharmaceutical compositions of the present invention, especially in reconstituted form, are useful for the treatment of pulmonary diseases, in particular, asthma, refractory asthma, chronic obstructive bronchitis, parenchymal, fibrotic and interstitial lung diseases and inflammations, and preferably for the prevention and treatment of acute and chronic organ transplant rejection reactions after lung transplantations and the diseases resulting therefrom such as bronchiolitis obliterans.

In preferred embodiments, however, the lyophilized pharmaceutical compositions as described in detail above in connection with the first aspect of the present invention are useful as medicaments for pulmonary application. The pulmonary application may be carried out after reconstitution or, more specifically, after dispersion of the lyophilized pharmaceutical composition as described above in connection with the first aspect of the invention in an aqueous carrier liquid, preferably in a sterile aqueous carrier liquid, to form a colloidal solution or dispersion, preferably to form a colloidal dispersion as described in further detail below.

In further preferred embodiments, the pulmonary application of the lyophilized pharmaceutical composition for use as described above is carried out by inhalation. In further preferred embodiments, the pulmonary application is carried out after conversion of the composition into an aerosol, such as by nebulization or aerosolization. After reconstitution or, more specifically, after dispersion in an aqueous carrier liquid, the lyophilized compositions according to the present invention may advantageously be aerosolized and administered by a nebulizer able to convert a solution, colloidal formulation or suspension such as the present compositions comprising liposomally solubilized CsA, into a high fraction of droplets which are able to reach the periphery of the lungs. Practically, a jet nebulizer, ultrasonic nebulizer, piezoelectric nebulizer, electro-hydrodynamic nebulizer, membrane nebulizer, electronic membrane nebulizer, or electronic vibrating membrane nebulizer may be used. Examples of suitable nebulizers include the SideStream® (Philips), AeroEclipse® (Trudell), LC Plus® (PARI), LC Star® (PARI), LC Sprint® (PARI), I-Neb® (Philips/Respironics), IH50 (Beurer), MicroMesh® (Health & Life, Schill), Micro Air® U22 (Omron), Multisonic® (Schill), Respimat® (Boehringer), eFlow® (PARI), AeroNebGo® (Aerogen), AeroNeb Pro® (Aerogen), and AeroDose® (Aerogen) device families.

Preferably however, a piezoelectric nebulizer, electro-hydrodynamic nebulizer, membrane nebulizer, electronic membrane nebulizer, or electronic vibrating membrane nebulizer may be used. In these cases, suitable nebulizers comprise the I-Neb® (Philips/Respironics), IH50 (Beurer), MicroMesh® (Health & Life, Schill), Micro Air® U22 (Omron), Multisonic® (Schill), Respimat® (Boehringer), eFlow® (PARI), AeroNebGo® (Aerogen), AeroNeb Pro® (Aerogen), and AeroDose® (Aerogen) device families.

In preferred embodiments, the pulmonal application of the lyophilized pharmaceutical composition in reconstituted form for use according to this aspect of the invention is carried out by means of an ultrasonic or electronic vibrating membrane nebulizer, preferably by means of a vibrating membrane nebulizer such as, for example, a device of the eFlow®, AeroNeb Pro or -Go or I-Neb type.

In further preferred embodiments, for targeting the drug CsA, especially in liposomally solubilized form as described above, to the lower respiratory tract, the composition for use according to this aspect of the present invention is aerosolized with an electronic vibrating membrane nebulizer. In a particularly preferred embodiment, the lyophilized pharmaceutical composition in reconstituted form for use according to the present invention is aerosolized with an eFlow® neblizer (PARI Pharma GmbH).

The eFlow® nebulizer nebulizes liquid drug formulations, such as the pharmaceutical compositions of the present invention in reconstituted form, with a perforated vibrating membrane resulting in an aerosol with a low ballistic momentum and a high percentage of droplets in a respirable size range, usually below 5 μm. The eFlow® is designed for a more rapid and efficient nebulization of medication due to a higher nebulization rate, lower drug wastage and a higher percentage of drug available as delivered dose (DD) and respirable dose (RD) compared to conventional nebulizers such as jet nebulizers.

In a third aspect, the present invention provides a kit for the preparation of an aqueous liposomal dispersion or solution, preferably a dispersion, especially a colloidal dispersion, for inhalation comprising a therapeutically effective amount of CsA in liposomally solubilized form, comprising
a lyophilized pharmaceutical composition according to the first aspect of the invention, and
a sterile aqueous carrier liquid.

The kit according to this aspect of the invention comprises as a first component the lyophilized pharmaceutical composition of the first aspect of the invention, namely a lyophilized pharmaceutical composition for reconstitution in an aqueous carrier liquid comprising:
a) liposome-forming structures comprising
   i. a therapeutically effective amount of cyclosporine A (CsA);
   ii. a membrane-forming substance selected from the group of phospholipids; and
   iii. a solubility-enhancing substance selected from the group of non-ionic surfactants; and
b) at least one disaccharide selected from the group consisting of saccharose, lactose and trehalose, wherein the at least one disaccharide is present in an amount of at least 40 wt.-% with regard to the total weight of the lyophilized composition.

The lyophilized pharmaceutical composition as described above may be provided or packaged in any suitable container that allows for sterile storage of the lyophilized composition over prolonged periods of time, such as 12 months or two years or even three years or even longer at or below 25° C., or, if necessary at reduced temperatures such as in the range from about 2° C. to about 8° C., and that allows for easy combination of the lyophilized composition with an aqueous carrier liquid. Accordingly, the kit of this aspect of the invention comprises as the second component an aqueous carrier liquid, preferably a sterile aqueous carrier liquid.

The lyophilized composition according to the first aspect of the present invention may, for example, be filled into single- or multiple-dose containers, preferably under aseptic conditions.

In specific embodiments, the lyophilized composition of the first aspect of the invention may be provided in sterile vials, preferably glass vials, wherein the vials preferably hold aliquots of the lyophilized pharmaceutical composition, especially as suitable and necessary for the pulmonal administration by inhalation as described above. In exemplary embodiments, the kit according to this aspect of the present invention comprises as the first component aliquots of the lyophilized pharmaceutical composition in an amount in the range of from about 150 mg to about 400 mg, preferably from about 150 to about 200 mg or from about 350 about 400 mg corresponding to an amount of about 5 mg or to an amount of about 10 mg of CsA per aliquot or unit or, in other words, to a CsA content of about 2.7 wt.-% of the total lyophilized pharmaceutical composition.

In preferred embodiments, the aqueous carrier liquid may be water or an aqueous solution of pharmaceutically acceptable salts or isotonizing agents and preferably may be sterile. In preferred embodiments, however, the sterile aqueous carrier liquid is an aqueous sodium chloride solution, preferably with the sodium chloride content of all 0.25% (w/v). Furthermore, the sterile aqueous carrier liquid may further comprise one or more buffer agents, preferably as described above in connection with the lyophilized pharmaceutical compositions according to the first aspect of the invention. Preferably, the sterile aqueous carrier liquid, especially the aqueous sodium chloride solution has a pH-well you in the range of from 4.0 to 7.0 and an osmolality in the range of from about 60 to about 100 mOsmol/kg.

Advantageously, the sterile aqueous carrier liquid is provided in an amount or in amounts suitable for the preparation of an aqueous liposomal dispersion for inhalation comprising CsA in liposomally solubilized form when combined with the amount or aliquot of the lyophilized pharmaceutical composition provided with the kit according to this aspect of the invention. In exemplary embodiments, the sterile aqueous carrier liquid, especially the aqueous sodium chloride solution as described above is provided in an amount of about 1.10 to about 1.50 mL to be combined with an aliquot of about 185 mg of the lyophilized pharmaceutical composition containing about 2.7 wt.-% of CsA (corresponding to 5 mg of CsA). In further exemplary embodiments, the sterile aqueous carrier liquid, especially the aqueous sodium chloride solution as described above is provided in an amount of about 2.20 to about 2.80 mL to be combined with an aliquot of about 375 mg of the lyophilized pharmaceutical composition containing about 2.7 wt.-% of CsA (corresponding to 10 mg of CsA).

In further specific embodiments, however, the sterile aqueous carrier liquid, more specifically the sterile aqueous sodium chloride solution as the second component of the present kit does not comprise any further excipients, buffers or chelating agents in addition to such constituents which are already comprised by the lyophilized pharmaceutical composition.

By combination of the first and the second component of the kit according to this aspect of the present invention it is possible to generate an aqueous dispersion of liposomes comprising CsA in liposomally solubilized form (L-CsA). In preferred embodiments, the aqueous dispersion of liposomes comprising CsA in liposomally solubilized form is a colloidal solution as described in further detail below.

Accordingly, in a fourth aspect the present invention provides a process for the preparation of an aqueous liposomal dispersion for inhalation comprising CsA in liposomally solubilized form by reconstitution of the lyophilized pharmaceutical composition of the first aspect of the invention as described above, comprising dispersing the lyophilized pharmaceutical composition for reconstitution in an aqueous carrier liquid, preferably in a sterile aqueous carrier liquid.

More specifically, the process according to this aspect of the invention comprises the steps of:
providing a lyophilized pharmaceutical composition comprising
   a) liposome-forming structures comprising
      i. a therapeutically effective amount of cyclosporine A (CsA);
      ii. a membrane-forming substance selected from the group of phospholipids; and
      iii. a solubility-enhancing substance selected from the group of non-ionic surfactants; and
   b) at least one disaccharide selected from the group consisting of saccharose, lactose and trehalose, wherein the at least one disaccharide is present in an amount of at least 40 wt.-% with regard to the total weight of the lyophilized composition; and
dispersing said lyophilized pharmaceutical composition in a sterile aqueous carrier liquid.

According to the first step, the process according to this aspect of the invention comprises the step of providing the lyophilized pharmaceutical composition of the first aspect of the invention comprising CsA in liposomally solubilized form, more specifically incorporated or intercalated in the liposome-forming structures as described in detail above. According to the second step of the process, said lyophilized pharmaceutical composition is dissolved or, more specifically, dispersed in a sterile aqueous carrier liquid, preferably in a sterile aqueous carrier liquid as described above in connection with the kit of the present invention. Accordingly, in preferred embodiments of this process of the present invention, the sterile aqueous carrier liquid may be water or may be, for example, an aqueous sodium chloride solution, preferably with a concentration of about 0.25% (w/v).

The amount of the lyophilized pharmaceutical composition may be chosen as required to achieve the desired and therapeutically effective amount and concentration of CsA after dispersion in the sterile aqueous carrier liquid. In exemplary embodiments, as discussed above in connection with the kit according to the third aspect of the present invention, specific aliquots such as aliquots of about 150 mg to about 400 mg or more specifically of about 185 mg or of about 375 mg of the lyophilized composition of the first aspect of the invention with an exemplary content of cyclosporine A of about 2.7 wt.-% (i.e. with a CsA content in the range of about 5 to about 10 mg or with regard to the specific amount with a CsA content of about 5 mg or about 10 mg) may be dispersed in aliquots of sterile aqueous carrier liquid such as saline with a volume in the range of from about 1.10 to about 2.80 mL or more specifically from 1.10 to about 1.50 mL or from about 2.20 to about 2.80 mL.

In general, however the amount of the lyophilized pharmaceutical composition comprising CsA as well as the amount of the sterile aqueous carrier liquid each may be chosen from broad ranges as appropriate to generate an aqueous liposomal dispersion for inhalation comprising cyclosporine A in liposomally solubilized form in a concentration that allows for the administration, especially for the administration by inhalation of the nebulized or aerosolized dispersion whereby a therapeutically effective amount of CsA may be delivered to the target tissue or organ, especially the lungs of the patient. In preferred embodiments, the resulting aqueous liposomal dispersion for inhalation comprising cyclosporine A in liposomally solubilized form comprises liposomally solubilized CsA (L-CsA) at a concentration in the range of from about 1 mg/mL to about 10 mg/mL, preferably from about 2 mg/mL to about 8 mg/mL, more preferably from 2.5 mg/mL to about 6 mg/mL, even more preferably from 3 mg/mL to about 4 mg/mL, especially at a concentration of about 4 mg/mL.

The step of dissolving or dispersing the lyophilized pharmaceutical compositions of the present invention in the sterile aqueous carrier liquid usually is readily achieved by contacting the lyophilized pharmaceutical composition with the chosen sterile aqueous carrier liquid either by adding the carrier liquid to the lyophilized composition or vice versa, preferably however, by adding the sterile aqueous carrier liquid in the chosen amount to the chosen amount of the lyophilized composition. If necessary at all, the dissolution or dispersion step may be supported or accelerated by gentle stirring or shaking, usually without the need for further mixing devices or equipment. Usually, the step of dissolving the lyophilized pharmaceutical composition of the invention in the sterile aqueous carrier is completed within short periods of time, such as up to 600 s, preferably up to 400 s or shorter, such as up to 300 s.

Accordingly, the present invention also provides a lyophilized pharmaceutical composition such as the composition according to the first aspect of the invention or a lyophilized pharmaceutical composition obtained or obtainable by the process for the preparation thereof as described in further detail below, wherein the lyophilized pharmaceutical composition may be dissolved in an aqueous carrier liquid within short periods of time, such as up to 600 sec, preferably up to 400 sec or shorter, such as up to 300 sec.

In a fifth aspect, the present invention provides a liquid liposomal dispersion comprising an aqueous carrier liquid and a therapeutically effective amount of CsA in liposomally solubilized form, prepared by a process comprising dispersing the lyophilized pharmaceutical composition of the first aspect of the invention in an aqueous carrier liquid, preferably in a sterile aqueous carrier liquid. More specifically, according to this aspect, the invention relates to a liquid liposomal dispersion comprising an aqueous carrier liquid and a therapeutically effective amount of cyclosporine A in liposomally solubilized form, which is prepared or obtained by the process of the fourth aspect of the invention as described in detail above comprising the steps of:
providing a lyophilized pharmaceutical composition comprising
   a) liposome-forming structures comprising
      i. a therapeutically effective amount of cyclosporine A (CsA);
      ii. a membrane-forming substance selected from the group of phospholipids; and
      iii. a solubility-enhancing substance selected from the group of non-ionic surfactants; and
   b) at least one disaccharide selected from the group consisting of saccharose, lactose and trehalose, wherein the at least one disaccharide is present in an amount of at least 40 wt.-% with regard to the total weight of the lyophilized composition; and
dispersing said lyophilized pharmaceutical composition in a sterile aqueous carrier liquid.

In preferred embodiments, the amounts of the lyophilized pharmaceutical composition and the aqueous carrier liquid may be chosen in the ranges as exemplarily described above. In further preferred embodiments, the amount of the lyophilized pharmaceutical composition comprising the at least one disaccharide selected from the group consisting of saccharose, lactose and trehalose in an amount of at least 40 wt.-% with regard to the total weight of the lyophilized composition and the amount of the aqueous carrier liquid may be chosen so that the resulting liquid liposomal dispersion has a content of the at least one disaccharide selected from the group consisting of saccharose, lactose and trehalose in the range of from about 5 to about 15 wt.-%, preferably in the range of from about 7.5 to about 12.5 wt.-%, based on the total weight of the resulting liquid liposomal dispersion.

In particularly preferred embodiments, at least one disaccharide selected from the group consisting of saccharose, lactose and trehalose is present in the resulting liquid liposomal dispersion in an amount in the range of from about 5 to about 10 wt.-% or from about 7.5 to about 10 wt.-%, or in an amount of about 7.5 wt.-% or about 10 wt.-%, all based on the total weight of the liquid liposomal dispersion. Surprisingly, it has been found that the resulting liposomal dispersions comprising CsA in liposomally solubilized form show characteristics equivalent or comparable to liquid dispersions of liposomally solubilized CsA (L-CsA) that has been prepared without prior lyophilization of the liposomes solubilizing the CsA or in the absence of a disaccharide, as described in further detail below.

In specific embodiments, the liquid liposomal dispersion comprising an aqueous carrier liquid and a therapeutically effective amount of CsA in liposomally solubilized form, prepared by a process comprising dispersing the lyophilized pharmaceutical composition of the first aspect of the invention in an aqueous carrier liquid as described above is an opalescent dispersion or solution. In further embodiments, the liquid liposomal dispersion is essentially free from visible particles. The liposomes comprised by said dispersion preferably have an average diameter or, more specifically, a z-average diameter of at most about 100 nm as measured by photon correlation spectroscopy using a Malvern ZetaSizer. Preferably, the liquid liposomal dispersion comprises liposomes with a z-average diameter as measured by photon correlation spectroscopy (Malvern ZetaSizer) in the range of from about 40 to about 100 nm and even more preferably in the range of from about 40 to about 70 nm.

In further specific embodiments, the liquid liposomal dispersion according to this aspect of the invention has a polydispersity index (PI) as measured by photon correlation spectroscopy of up to about 0.50, preferably of up to about 0.4 and even more preferably in the range of from about 0.1 to about 0.3.

In further embodiments, the liquid liposomal dispersions according to this aspect of the invention have an osmolality in the range of from about 300 to about 550 mOsmol/kg, preferably in the range of from about 430 to about 550 mOsmol/kg. The pH-value of the liquid liposomal dispersions according to this aspect of the invention preferably is in the range of from about 6.0 to 7.0. In further embodiments, after 1:10 dilution, the liquid liposomal dispersion according to this aspect of the invention has a turbidity of up to 200 NTU (Nephelometric Turbidity Units).

It has surprisingly been found that the liquid liposomal dispersion according to this aspect of the invention which have been prepared by dispersing the lyophilized pharmaceutical composition of the first aspect of the present invention comprising a disaccharide selected from the group consisting of saccharose, lactose and trehalose, in an amount of at least 40 wt.-% in an aqueous carrier liquid comprise liposomes that are equal in size or only slightly larger compared to the liposomes in a corresponding dispersion prior to the lyophilization as described below. Accordingly, the present invention according to this aspect provides liquid liposomal aqueous dispersions comprising liposomes with a median diameter measured as the z-average diameter as measured by photon correlation spectroscopy (Malvern ZetaSizer) which is equal or up to 20% larger, preferably only up to 10% larger than the z-average diameter of the liposomes used to prepare the lyophilized pharmaceutical composition of the present invention prior to lyophilization, preferably which is equal or up to 20% larger than the liposomes formed by a process according to process for preparation according to the sixth aspect of the invention before lyophilization.

In a sixth aspect, the present invention provides a process for the preparation of a lyophilized pharmaceutical composition comprising a therapeutically effective amount of CsA in liposomally solubilized form for reconstitution in an aqueous carrier liquid, preferably for the preparation of the lyophilized pharmaceutical composition of the first aspect of the invention, wherein the at least one disaccharide selected from the group consisting of saccharose, lactose and trehalose is present in an amount of at least 40 wt.-% with regard to the total weight of the lyophilized composition, the process comprising the steps:
(a) providing a liquid aqueous dispersion of liposomes, said liposomes comprising:
   i. a therapeutically effective amount of cyclosporine A (CsA);
   ii. a membrane-forming substance selected from the group of phospholipids; and
   iii. a solubility-enhancing substance selected from the group of non-ionic surfactants; and optionally
   iv. one or more further excipients, such as buffers and/or chelating agents;
wherein said liquid aqueous dispersion further comprises at least one disaccharide selected from the group consisting of saccharose, lactose and trehalose, which is present in an amount of at least 40 wt.-% with regard to the total weight of the lyophilized composition; and
(b) lyophilizing said aqueous dispersion.

The process according to this aspect of the invention is useful for the preparation of the lyophilized pharmaceutical compositions according to the first aspect of the inventions comprising a therapeutically effective amount of CsA in liposomally solubilized form. In the first step (a) of this aspect of the invention, a liquid aqueous dispersion of liposomes or in other words a dispersion of liposomes in an aqueous carrier liquid as described in connection with the other aspects of the present invention above is prepared or provided. Said liquid aqueous dispersion of liposomes may be provided by mixing the chosen amount of CsA, the membrane-forming substance selected from the group phospholipids and the solubility-enhancing substance selected from the group of non-ionic surfactants and optionally one or more further excipients in a suitable aqueous carrier liquid such as water or saline as described above. The liquid aqueous dispersion further comprises at least one disaccharide selected from the group consisting of saccharose, lactose and trehalose, preferably saccharose, which is present in an amount of at least 40 wt.-% with regard to the total weight of the final lyophilized composition.

The disaccharide may be added to the chosen aqueous carrier liquid before, together with or after the addition of the other components of the aqueous liquid dispersion. In preferred embodiments however, the chosen disaccharide, preferably saccharose, is added and dissolved in the aqueous carrier liquid before the other components are added to the mixture, especially before the resulting mixture is exposed to homogenization conditions as described below. This may ensure that the formation of liposomes occurs in the presence of the chosen disaccharide or, more specifically, in the aqueous solution of the chosen disaccharide so that the disaccharide may also present or enclosed in the inner lumen of the liposomes to be formed or may also be incorporated or intercalated in the bilayer membranes to be formed. In specific embodiments the chosen disaccharide may be present in the inner lumen of the liposomes at the same concentration as in the surrounding continuous phase of the liquid aqueous dispersion.

In the second step (b), the liquid aqueous dispersion of liposomes as formed in the first step (a) as described above is then lyophilized as described in further detail below.

In specific embodiments, the present invention provides a process for the preparation of a lyophilized pharmaceutical composition comprising a therapeutically effective amount of CsA in liposomally solubilized form for reconstitution in an aqueous carrier liquid, preferably for the preparation of the lyophilized pharmaceutical composition of the first aspect of the invention wherein the at least one disaccharide selected from the group consisting of saccharose, lactose and trehalose is present in an amount of at least 40 wt.-% with regard to the total weight of the lyophilized composition, the process comprising the steps:
(a1) providing an aqueous mixture comprising:
   at least one disaccharide selected from the group consisting of saccharose, lactose and trehalose;
   a membrane-forming substance selected from the group of phospholipids;
   a therapeutically effective amount of cyclosporine A (CsA); and
   a solubility-enhancing substance selected from the group of non-ionic surfactants; and optionally
   one or more further excipients
   and
(a2) exposing the aqueous mixture to homogenization conditions; and
(b1) lyophilizing the resulting homogenized mixture to form the lyophilized pharmaceutical composition.

As already mentioned above, the aqueous mixture to be provided according to step (a1) further comprises a suitable aqueous carrier liquid or vehicle, preferably water or saline, in which the other components of the aqueous mixture may be dissolved or dispersed. In general, the aqueous mixture may be prepared in any suitable way, for example by providing the aqueous carrier liquid, preferably water, and adding the other components as listed above. Alternatively, the other components may be provided first and the chosen aqueous carrier liquid may be added afterwards. In preferred embodiments, however, the chosen aqueous liquid carrier, preferably water, is provided and the other components as named above are added, preferably in consecutive manner. The resulting mixture may then be stirred by standard techniques to form the corresponding solutions or dispersions, preferably a homogeneous dispersion as necessary.

In a second step (a2) the resulting aqueous mixture, preferably the resulting homogeneous dispersion is then exposed to homogenization conditions to generate a colloidal dispersion of cyclosporine A in liposomally solubilized form. In preferred embodiments, said homogenization conditions comprise high-pressure homogenisation, as known to those of skill in the art, for example by using a Microfluidics M-110EH. The high-pressure homogenization may be carried out a single time or several times repeatedly. Preferably, high-pressure homogenization is carried out repeatedly, such as about 5 to about 15 times. Furthermore, high-pressure homogenization may be carried out at any suitable pressure, usually at pressures of up to about 1500 bar, or at pressures in the range of from about 50 to about 1500 bar. Preferably, high-pressure homogenization may be carried out repeatedly, such as about 5 to about 15 times at pressures in the range of from about 100 to about 1000 bar, if necessary under reduced pressure.

In a third step (b1) comprises lyophilizing the resulting homogenized mixture, preferably the resulting homogeneous colloidal dispersion to form the lyophilized pharmaceutical composition. The lyophilization can be conducted according to standard techniques known to those of skill in the art, for example by using a LyoStar MNL-055-A/LSACC3E. The lyophilization to form the lyophilized pharmaceutical compositions of the invention may be conducted in continuous manner, for example at constant pressure and temperature or preferably may be conducted stepwise, wherein each step of the lyophilization protocol or process may be conducted at specific pressures, temperatures and for a defined duration. In exemplary embodiments, the lyophilization process or cycle may comprise up to 20, or from about 2 to about 15, preferably from about 5 to about 15 consecutive steps. Each step may, for example, be conducted at temperature within the range of from about 40° C. to about −60° C., preferably from about 20° C. to about −50° C., either at a constant temperature or at temperatures that may be raised or lowered at a certain gradient. Furthermore, each lyophilization step may be conducted at reduced pressures, for example at pressures below ambient pressure, such as in the range from about 0.005 mbar to about 800 mbar, preferably from about 0.009 mbar to about 0.500 mbar, or to about 0.400 mbar or 0.300 mbar.

In a preferred embodiment, the process for the preparation of a lyophilized pharmaceutical composition of this aspect of the invention comprises the steps of:
providing an aqueous solution of at least one disaccharide selected from the group consisting of saccharose, lactose and trehalose, preferably wherein wherein the at least one disaccharide is present in an amount of at least 40 wt.-% with regard to the total weight of the lyophilized composition;
adding a membrane-forming substance selected from the group of phospholipids to form a first intermediate mixture, preferably in form of a homogeneous dispersion;
adding cyclosporine A to the first intermediate mixture to form a second intermediate mixture;
adding a solubility-enhancing substance selected from the group of non-ionic surfactants to the second intermediate mixture to form a third intermediate mixture;
exposing the resulting third intermediate mixture to homogenization conditions, preferably to form a homogeneous liposomal dispersion; and
lyophilizing the resulting homogenized mixture, preferably the resulting homogeneous liposomal dispersion to form the lyophilized pharmaceutical composition.

The lyophilization as described above may generally be performed with any amounts of the resulting homogenized mixture. Preferably, however, the resulting homogenized mixture is portioned or divided in aliquots and filled and lyophilized in the final packaging, preferably the glass vials.

In further preferred embodiments, the process as described above further comprises the step of
sterilizing the intermediate mixture, preferably following the step of exposing the resulting intermediate mixture to homogenization conditions.

In further preferred embodiments, the optional step of sterilization comprises sterile filtration, for example by using a sterile filter, preferably with pore sizes of about 0.2 μm.

In a seventh aspect, the present invention provides a lyophilized pharmaceutical composition obtainable by a process comprising the steps of:
(a) providing a liquid aqueous dispersion of liposomes, said liposomes comprising:
   i. a therapeutically effective amount of cyclosporine A (CsA);
   ii. a membrane-forming substance selected from the group of phospholipids; and
   iii. a solubility-enhancing substance selected from the group of non-ionic surfactants; and optionally
   iv. one or more further excipients, such as buffers and/or chelating agents;
wherein said liquid aqueous dispersion further comprises at least one disaccharide selected from the group consisting of saccharose, lactose and trehalose, which is present in an amount of at least 40 wt.-% with regard to the total weight of the lyophilized composition; and
(b) lyophilizing said aqueous dispersion.

The present aspect of the invention refers to a lyophilized pharmaceutical composition obtainable or obtained by a process according to the sixth aspect of the invention as described in detail above. It should be noted, that the lyophilized pharmaceutical composition obtainable by the process as outlined above corresponds to the lyophilized pharmaceutical composition according to the first aspect of the invention. Accordingly, all features, properties, advantages and technical effects as described for the lyophilized pharmaceutical composition of the first aspect of the invention as well as to the process for the manufacture thereof according to the sixth aspect of the invention also apply to the present lyophilized pharmaceutical composition which are obtainable by the process for the preparation thereof as described above, either as such or in combination with each other.

Accordingly, in specific embodiments the present invention also refers to a lyophilized pharmaceutical composition obtainable by a process comprising the steps of:
(a1) providing an aqueous mixture comprising:
   at least one disaccharide selected from the group consisting of saccharose, lactose and trehalose;
   a membrane-forming substance selected from the group of phospholipids;
   a therapeutically effective amount of cyclosporine A (CsA); and
   a solubility-enhancing substance selected from the group of non-ionic surfactants;
   and
(a2) exposing the aqueous mixture to homogenization conditions; and
(b1) lyophilizing the resulting homogenized mixture to form the lyophilized pharmaceutical composition.

More specifically, the present invention also refers to a lyophilized pharmaceutical composition obtainable by a process comprising the steps of:
providing an aqueous solution of at least one disaccharide selected from the group consisting of saccharose, lactose and trehalose, preferably wherein wherein the at least one disaccharide is present in an amount of at least 40 wt.-% with regard to the total weight of the lyophilized composition;
adding a membrane-forming substance selected from the group of phospholipids to form a first intermediate mixture, preferably in form of a homogeneous dispersion;
adding CsA to the first intermediate mixture to form a second intermediate mixture;
adding a solubility-enhancing substance selected from the group of non-ionic surfactants to the second intermediate mixture to form a third intermediate mixture;
exposing the resulting third intermediate mixture to homogenization conditions, preferably to form a homogeneous liposomal dispersion; and
lyophilizing the resulting homogenized mixture, preferably the resulting homogeneous liposomal dispersion to form the lyophilized pharmaceutical composition.

It should be noted that in the processes by which the lyophilized pharmaceutical composition may be obtained the formation of the aqueous dispersion of liposomes solubilizing CsA is conducted in an aqueous carrier liquid comprising the disaccharide selected from saccharose, lactose and trehalose, or, in other words, the liposomes are formed in the presence of the disaccharide. This, however, leads to the incorporation of the selected disaccharide in the inner lumen of the liposomes solubilizing CsA. In addition to the lyoprotectant effect of the disaccharide, this leads to the surprising and unique characteristics of the lyophilized pharmaceutical composition of the present invention, such as the surprising stability, both in lyophilized and in reconstituted form as well as to their surprising solubility.

The following is a list of numbered embodiments comprised by the present invention:

1. A lyophilized pharmaceutical composition for reconstitution in an aqueous carrier liquid comprising:
a) liposome-forming structures comprising
   i. a therapeutically effective amount of cyclosporine A (CsA);
   ii. a membrane-forming substance selected from the group of phospholipids; and
   iii. a solubility-enhancing substance selected from the group of non-ionic surfactants;
and
b) at least one disaccharide selected from the group consisting of saccharose, lactose and trehalose,
   wherein the at least one disaccharide is present in an amount of at least 40 wt.-% with regard to the total weight of the lyophilized composition.
   2. The composition according to item 1, wherein the at least one disaccharide is present in an amount selected in the range of from of 50 wt.-% to 80 wt.% with regard to the total weight of the lyophilized composition.
   3. The composition according to item 1or 2, wherein the at least one disaccharide is present in an amount selected in the range of from of 60 wt.-% to 75 wt. % with regard to the total weight of the lyophilized composition.
   4. The composition according to any preceding item, wherein the liposome forming structures comprise a bilayer membrane formed of the membrane-forming substance selected from the group of phospholipids.
   5. The composition according to any preceding item, wherein the liposome-forming structures are at least partly present in unilamellar form.
   6. The composition according to any preceding item, wherein the liposome-forming structures comprise an inner lumen surrounded or at least partially surrounded by the bilayer membrane formed of the membrane forming substance selected from the group of phospholipids.
   7. The composition according to any preceding item, wherein the inner lumen of the liposome-forming structures are at least partially dehydrated.
   8. The composition according to any preceding item, wherein the inner lumen of the liposome-forming structures contains (fractions of) the at least one disaccharide selected from the group consisting of saccharose, lactose and trehalose.
   9. The composition according to any preceding item, wherein the CsA is at least partially incorporated (or intercalated) in the bilayer membrane of the liposome-forming structures.
   10. The composition according to any preceding item, wherein the CsA is predominantly (for example by at least about 90% or even at least about 95% to about 97.5%) incorporated in the bilayer membrane of the liposome-forming structures.
   11. The composition according to any preceding item, wherein the composition comprises cyclosporine A in an amount of from 2 to 4 wt.-%, based on the weight of the lyophilized composition.
   12. The composition according to any preceding item, wherein the ratio of the weight of the at least one disaccharide to the weight of cyclosporine A in the lyophilized composition is selected in the range of from 10:1 to 30:1.
   13. The composition according to any preceding item, wherein the at least one disaccharide is saccharose and/or lactose.
   14. The composition according to any preceding item, wherein the at least one disaccharide is saccharose.
   15. The composition according to any preceding item, wherein the membrane-forming substance selected from the group of phospholipids is a mixture of natural phospholipids.
   16. The composition according to any preceding item, wherein the membrane-forming substance selected from the group of phospholipids is a lecithin containing unsaturated fatty acid residues.
   17. The composition according to any preceding item, wherein the membrane forming substance selected from the group of phospholipids is a lecithin selected from the group consisting of soy bean lecithin, Lipoid S75, Lipoid S100, Phospholipon® G90, 100 or a comparable lecithin.
   18. The composition according to any preceding item, wherein the content of the membrane-forming substance selected from the group of phospholipids is from about 10 to about 30 wt.-% and preferably from about 20 to about 30 wt.-%, based on the weight of the lyophilized composition.
   19. The composition according to any preceding item, wherein the content of the solubility-enhancing substance selected from the group of non-ionic surfactants may preferably be chosen in the range of from about 0.01 to about 5 wt.-%, or from about 0.1 to about 4 wt.-%, or from about 0.5 to about 3.5 wt.-%, or from about 1 to about 3 wt.-%, preferably from about 1.5 to about 2.5 wt.-%, based on the total weight of the lyophilized composition.
   20. The composition according to any preceding item, wherein the solubility-enhancing substance selected from the group of non-ionic surfactants is selected from the group of polysorbates.
   21. The composition according to any preceding item, wherein the solubility-enhancing substance selected from the group of non-ionic surfactants is polysorbate 80.
   22. The composition according to any preceding item, wherein the weight ratio of phospholipid to polysorbate is selected in the range of from 15:1 to 9:1, preferably between from about 14:1 to about 12:1, for example, about 13:1.
   23. The composition according to any preceding item, wherein the weight ratio between the (sum of the) phospholipid and the nonionic surfactant on the one hand and cyclosporine A on the other hand is selected in the range of from about 5:1 to about 20:1, preferably from about 8:1 to about 12:1 and more preferably about 10:1.
   24. The composition according to any preceding item, wherein the weight ratio between the phospholipid (lecithin), the nonionic surfactant and cyclosporine A is between about 15:1:1.5 and 5:0.3:0.5, and preferably at about 9:0.7:1.
   25. The composition according to any preceding item, wherein the amount of residual water comprised by the lyophilized composition is in the range of up to 2 wt.-%.
   26. The composition according to any preceding item, wherein the composition comprises one or more further excipients.
   27. The composition according to item 26, wherein the one or more further excipients are selected from buffers and chelating agents.
   28. The composition according to any preceding item, wherein the lyophilized composition essentially consists of cyclosporine A; the membrane-forming substance selected from the group of phospholipids; the solubility-enhancing substance selected from the group of non-ionic surfactants; the at least one disaccharide selected from the group consisting of saccharose, lactose and trehalose; and optionally further excipients and residual amounts of water.
   29. The lyophilized pharmaceutical composition of any preceding item for use as a medicament for pulmonary application.
   30. The lyophilized pharmaceutical composition for use according to item 29, wherein the pulmonary application is carried out after reconstitution (dispersion) of the lyophilized pharmaceutical composition of any of claims 1 to 29 in an aqueous carrier liquid to form a colloidal solution or dispersion.
   31. The lyophilized pharmaceutical composition for use according to item 29 or 30, wherein the pulmonary application is carried out after conversion of the composition into an aerosol, such as by nebulization.
   32. The lyophilized pharmaceutical composition for use according to any of items 29 to 31, wherein the pulmonary application is carried out by inhalation.
   33. The lyophilized pharmaceutical composition for use according to any of items 29 to 32, wherein the pulmonal application is carried out by means of an ultrasonic or electronic vibrating membrane nebulizer, preferably by means of a vibrating membrane nebulizer such as, for example, a device of the eFlow®, AeroNeb Pro or -Go or I-Neb type.
   34. The lyophilized pharmaceutical composition for use according to any of items 29 to 33 for the prophylaxis and treatment asthma, refractory asthma, chronic obstructive bronchitis, parenchymal, fibrotic and interstitial lung diseases and inflammations, and preferably for the prevention and treatment of acute and chronic organ transplant rejection reactions after lung transplantations and the diseases resulting therefrom such as bronchiolitis obliterans.
   35. A kit for the preparation of an aqueous liposomal dispersion for inhalation comprising a therapeutically effective amount of cyclosporine A in liposomally solubilized form, comprising
a lyophilized pharmaceutical composition according to any of items 1 to 28, and
an aqueous carrier liquid.
   36. The kit according to item 35, wherein the sterile aqueous carrier liquid is an aqueous sodium chloride solution.
   37. The kit according to item 35 or 36, wherein the sterile aqueous carrier liquid further comprises one or more buffer agents.
   38. The kit according to any of items 35 to 37, wherein the aqueous dispersion of liposomes comprising cyclosporine A in liposomally solubilized form is a colloidal solution.
   39. A process for the preparation of an aqueous liposomal dispersion for inhalation comprising cyclosporine A in liposomally solubilized form by reconstitution of the lyophilized pharmaceutical composition of any one of claims 1 to 28, comprising dispersing the lyophilized pharmaceutical composition according to any of items 1 to 28 in a sterile aqueous carrier liquid.
   40. The process according to item 39, wherein the sterile aqueous carrier liquid is an aqueous sodium chloride solution, preferably with a concentration of about 0.25% (w/v).
   41. The process according to item 39 or 40, wherein aliquots of 186 mg or 372 mg of the lyophilized composition of any of claims 1 to 20 is dispersed in aliquots of saline with a volume in the range of from 1.2 to 2.4 ml.
   42. The process according to any of items 39 to 41, wherein the step of dissolving the lyophilized pharmaceutical composition according to any of items 1 to 28 in the sterile aqueous carrier is completed within up to 300 s.
   43. A liquid liposomal dispersion comprising an aqueous carrier liquid and a therapeutically effective amount of cyclosporine A in liposomally solubilized form, prepared by a process comprising dispersing the lyophilized pharmaceutical composition of any of items 1 to 28 in an aqueous carrier liquid.
   44. The liquid liposomal dispersion of item 43, prepared or obtained by the process of any of claims 39 to 42.
   45. The liquid liposomal dispersion of item 43 or 44, wherein the at least one disaccharide selected from the group consisting of saccharose, lactose and trehalose is present in an amount of 5 to 15 wt.-%, based on the total weight of the liquid liposomal dispersion.
   46. The liquid liposomal dispersion according to any of items 43 to 45, wherein the at least one disaccharide selected from the group consisting of saccharose, lactose and trehalose is present in an amount of 7.5 to 12.5 wt.-%, based on the total weight of the liquid liposomal dispersion.
   47. The liquid liposomal dispersion according to any of items 43 to 46, wherein the dispersion has an osmolality in the range of from about 430 to about 550 mOsmol/kg.
   48. The liquid liposomal dispersion according to any of items 43 to 47, wherein the dispersion has a polydispersity index (PI) as measured by photon correlation spectroscopy up to about 0.50.
   49. The liquid liposomal dispersion according to any of items 43 to 48, wherein the dispersion is essentially free from visible particles.
   50. The liquid liposomal dispersion according to any of items 43 to 49, wherein the dispersion comprises liposomes with a z-average diameter as measured by photon correlation spectroscopy in the range of from about 40 to about 100 nm.
   51. The liquid liposomal dispersion according to any of items 43 to 50, wherein the dispersion comprises liposomes with a n the z-average diameter as measured by photon correlation spectroscopy which is equal or up to 20% larger than the z-average diameter of the liposomes used to prepare the lyophilized pharmaceutical composition of any of items 1 to 28 prior to lyophilization, preferably which is equal or up to 20% larger than the liposomes formed by a process according to item 53 before lyophilization.
   52. The liquid liposomal dispersion according to any of items 43 to 51, wherein the dispersion has a turbidity after 1:10 dilution of up to 200 NTU (Nephelometric Turbidity Units).
   53. A process for the preparation of a lyophilized pharmaceutical composition comprising a therapeutically effective amount of cyclosporine A in liposomally solubilized form for reconstitution in an aqueous carrier liquid, preferably for the preparation of the lyophilized pharmaceutical composition of any of items 1 to 28, the process comprising the steps:
(a) providing a liquid aqueous dispersion of liposomes, said liposomes comprising:
   i. a therapeutically effective amount of cyclosporine A (CsA);
   ii. a membrane-forming substance selected from the group of phospholipids; and
   iii. a solubility-enhancing substance selected from the group of non-ionic surfactants; and optionally
   iv. one or more further excipients, such as buffers and/or chelating agents;
      wherein said liquid aqueous dispersion further comprises at least one disaccharide disaccharide selected from the group consisting of saccharose, lactose and trehalose, which is present in an amount of at least 40 wt.-% with regard to the total weight of the lyophilized composition; and
(b) lyophilizing said aqueous dispersion.
   54. The process according to item 53, comprising the steps:
(a1) providing an aqueous mixture comprising:
   at least one disaccharide selected from the group consisting of saccharose, lactose and trehalose;
   a membrane-forming substance selected from the group of phospholipids;
   a therapeutically effective amount of cyclosporine A (CsA); and
   a solubility-enhancing substance selected from the group of non-ionic surfactants; and optionally
   one or more further excipients
   and
(a2) exposing the aqueous mixture to homogenization conditions; and
(b1) lyophilizing the resulting homogenized mixture to form the lyophilized pharmaceutical composition.
   55. The process according to item 53 or 54, comprising the steps:
providing an aqueous solution of at least one disaccharide selected from the group consisting of saccharose, lactose and trehalose;
adding a membrane-forming substance selected from the group of phospholipids to form a first intermediate mixture, preferably in form of a (homogeneous) dispersion;
adding cyclosporine A to the first intermediate mixture to form a second intermediate mixture;
adding a solubility-enhancing substance selected from the group of non-ionic surfactants to the second intermediate mixture to form a third intermediate mixture;
exposing the resulting third intermediate mixture to homogenization conditions; and
lyophilizing the resulting homogenized mixture to form the lyophilized pharmaceutical composition.
   56. The process according to any of items 53 to 55, further comprising the step of
sterilizing the intermediate the mixture, preferably following the step of exposing the resulting intermediate mixture to homogenization conditions.
   57. The process according to item 56, wherein the step of sterilization comprises sterile filtration.
   58. The process according to any of items 54 to 57, wherein the homogenization conditions comprise high-pressure homogenisation.
   59. The process according to any of items 54 to 58, wherein the high-pressure homogenization is carried out repeatedly.
   60. The process according to any of items 54 to 59, wherein the high-pressure homogenization is carried out about 5 to about 15 times.
   61. The process according to any of items 54 to 60, wherein the high-pressure homogenization is carried out at a pressure in the range of from 100 to 1000 bar.
   62. The process according to any of items 53 to 61, wherein the at least one disaccharide is present in an amount of at least 40 wt.-% with regard to the total weight of the lyophilized composition.
   63. A lyophilized pharmaceutical composition obtainable by a process comprising the steps of
(a) providing a liquid aqueous dispersion of liposomes, said liposomes comprising
   i. a therapeutically effective amount of cyclosporine A (CsA);
   ii. a membrane-forming substance selected from the group of phospholipids; and
   iii. a solubility-enhancing substance selected from the group of non-ionic surfactants; and optionally
   iv. one or more further excipients, such as buffers and/or chelating agents.
   wherein said liquid aqueous dispersion further comprises at least one disaccharide selected from the group consisting of saccharose, lactose and trehalose, which is present in an amount of at least 40 wt.-% with regard to the total weight of the lyophilized composition; and
(b) lyophilizing said aqueous dispersion.
   64. A lyophilized pharmaceutical composition obtained or obtainable by a process according to any of items 53 to 62.

The following examples serve to illustrate the present invention without, however, limiting it in any respect:

### EXAMPLES

### Example 1: Preparation of a Lyophilized Pharmaceutical Composition Comprising Cyclosporine A (5 mg) in Liposomally Solubilized Form

### 1.1 Step 1: Preparation of Liposomal Solution:

1.1.1 Approximately 70% (˜104 L) water for injections was filled into the preparation vessel. It was degassed by introduction of nitrogen gas and warmed up to a temperature of 40 to 45° C. 18.0 kg of saccharose, 450.0 g of sodium dihydrogen phosphate dihydrate, 612.0 g of disodium hydrogen phosphate decahydrate and 36.0 g of disodium edetate were added together and approximately 5% (8.0 L) of water for injections were used for rinsing. The mixture was stirred until a visually clear solution was obtained. The solution was cooled down to 20 to 25° C. and 6480.0 g of soy bean lecithin Lipoid S100 was added and stirred until a homogenous dispersion was obtained. Then, 504.0 g of polysorbate 80 HP (Tween 80) was added under gentle stirring to avoid foaming and the container holding the polysorbate was rinsed with approximately 100 mL of water for injections. After that, 720.0 g of cyclosporine and approximately 5% (8 L) of water for injections was added and the mixture was stirred until a homogenous dispersion was formed.

1.1.2 Following that, the resulting dispersion was cooled to a temperature of 5 to 10° C. and exposed to high pressure homogenization at a pressure of 100 bar (first stage) and 1000 bar (second stage), respectively, using a GEA high pressure homogenizer. The high-pressure homogenization was repeated 9 times (cycles).

1.1.3 The resulting homogenized suspension was then filtered through a bioburden reduction filter with a pore size of 0.2 μm in minimum once and transferred into a filling/storage tank.

### 1.2 Step 2: Aseptic Filling, Lyophilization and Packaging

1.2.1 Glass vials with a filling volume of 10 mL were sterilized in a hot-air sterilizing tunnel, cooled down and filled with aliquots of 1.35 mL (5 mg dosage) of the dispersion as prepared according to step 1 as described above after aseptic sterilisation using two sterile filters with a pore size of 0.2 μm between the filling/storage tank and the filling needles. The vials were then partially closed with sterilized lyophilization stoppers and loaded into a lyophilizer (GEA Lyovac FCM) and were lyophilized according to a 72 h lyophilization cycle.

1.2.2 After completion of lyophilization, the vials were automatically fully stoppered in the lyophilization chamber. The vials were unloaded and closed with flip-tear-off caps. Each vial contained approximately 190 mg of an almost white, homogenous, porous lyophilization cake containing 5 mg of cyclosporine A in liposomally solubilized form with a maximum residual moisture of 2% (w/w) and a shelf life of 3 years.

1.2.3 The composition of the lyophilized drug product prepared as described above is summarized in Table 1 below:

TABLE 1

| | | |
|---|---|---|
| Ingredient | Quantity per unit | Quantity % (w/w) |

| | | | |
|---|---|---|---|
| Cyclosporine A | 5 | mg | 2.69 |
| Polysorbate 80 | 3.5 | mg | 1.88 |
| Lipoid S100 | 45 | mg | 24.18 |
| Sucrose | 125 | mg | 67.16 |
| Sodium dihydrogen | 3.125 | mg | 1.68 |
| phosphate dihydrate | | | |
| Disodium hydrogen | 4.25 | mg | 2.28 |
| phosphate | | | |
| dodecahydrate | | | |
| Disodium edetate | 0.25 | mg | 0.13 |
| dihydrate | | | |
| Total | 186.125 | mg | 100.00 |

### Example 2: Reconstitution of the Lyophilized Composition Comprising Cyclosporine A to Yield a Colloidal Solution of Liposomally Solubilized Cyclosporine A for Nebulization and Inhalation

2.1 To an aliquot of 186.1 mg of the lyophilization cake as prepared according to Example 1 above containing 5 mg of cyclosporine A was added 1.35 ml of a sterile aqueous sodium chloride solution with a concentration of 0.25% (w/v) to give an opalescent aqueous solution of liposomal cyclosporine A for inhalation purposes with a concentration of CsA of 4 mg/mL.

2.2 For the preparation of a corresponding colloidal solution with a content of liposomally solubilized cyclosporine A of 10 mg, an aliquot of 372.3 mg of the lyophilization cake as prepared according to Example 1 above was dissolved in 2.65 mL of a sterile aqueous sodium chloride solution with a concentration of 0.25% (w/v) to give an opalescent aqueous solution of liposomal cyclosporine A for inhalation purposes with a concentration of CsA of 4 mg/mL.

2.3 The composition of the reconstituted drug product prepared as described above is summarized in Table 2 below:

TABLE 2

| | | |
|---|---|---|
| Ingredient | Quantity per unit | Quantity % (w/v) |

| | | | |
|---|---|---|---|
| Cyclosporine A | 5 | mg | 0.4 |
| Polysorbate 80 | 3.5 | mg | 0.28 |
| Lipoid S100 | 45 | mg | 3.6 |
| Sucrose | 125 | mg | 10 |
| Sodium dihydrogen | 3.125 | mg | 0.25 |
| phosphate dihydrate | | | |
| Disodium hydrogen | 4.25 | mg | 0.34 |
| phosphate | | | |
| dodecahydrate | | | |
| Disodium edetate | 0.25 | mg | 0.02 |
| dihydrate | | | |
| Sodium chloride | 2.8 | mg | 0.22 or 0.23 |

| | | |
|---|---|---|
| Water for Injection | Ad 1.25 mL | Ad 100 |

### Example 3: Preparation of Lyophilized Compositions Comprising CsA in Liposomally Solubilized Form and Reconstituted Liposomal Solutions Thereof in the Presence of Lactose or Trehalose

3.1 Following the protocols of Example 1 and Example 2 above, lyophilized compositions comprising CsA in liposomally solubilized form were prepared in the presence of trehalose as the disaccharide and in the presence of lactose monohydrate. Both disaccharides were used in amount necessary to obtain a content of the respective sugar of 7.5 and 10% (w/v) in the final reconstituted liposomal solution. Furthermore, in addition to the composition summarized in Table 3 above, corresponding liposomal solutions with a content of saccharose of 5.0 and 7.5% (w/v) were prepared. In all cases opalescent colloidal solutions were obtained with a polydispersity index (PI) and liposome diameters (measured as the z-average diameter, ZAve) as summarized in Table 3 below:

TABLE 3

| | Content | ZAve | | Opalescent |
|---|---|---|---|---|
| Disaccharide | [% (w/v)] | [nm] | PI | solution |
| Trehalose | 7.5 | 123.2 | 0.344 | Yes |
| Trehalose | 10 | 103.5 | 0.181 | Yes |
| Lactose-monohydrate | 7.5 | 89.1 | 0.115 | Yes |
| Lactose-monohydrate | 10 | 88.1 | 0.101 | Yes |
| Saccharose | 5 | 93.0 | 0.346 | Yes |
| Saccharose | 7.5 | 86.1 | 0.445 | Yes |
| Saccharose | 10 | 47.7 | 0.253 | Yes |

### Example 4: Comparison of Characteristics of Aqueous Liposomal Dispersions Comprising CsA in Liposomally Solubilized Form before Lyophilization and after Reconstitution of the Lyophilisate

4.1 An aqueous dispersion of liposomally solubilized CsA comprising 10% (w/v) of saccharose was prepared as described in Example 1, step 1. Likewise, an aqueous dispersion of liposomally solubilized CsA comprising 10% (w/v) of lactose was prepared. Furthermore, the aqueous dispersion comprising 10% (w/v) of saccharose was lyophilized as described in Example 1, step 2, and reconstituted as described in Example 2. Key characteristics of the resulting dispersions are summarized in Table 4 below:

TABLE 4

| | | ZAve | | Omolality |
|---|---|---|---|---|
| Dispersion comprising CsA | pH | [nm] | PI | [osmol/kg] |
| 10% (w/v) saccharose; before | 6.62 | 51.0 | 0.262 | 0.436 |
| lyophilization/reconstitution | | | | |
| 10% (w/v) saccharose; after | 6.64 | 55.3 | 0.230 | 0.383 |
| lyophilization/reconstitution | | | | |
| 10% (w/v) lactose; before | 6.58 | 50.5 | 0.251 | 0.433 |
| lyophilization/reconstitution | | | | |

### Example 5: Stability of the Lyophilized Compositions Comprising Liposomally Solubilized CsA; Comparison of Stabilities

### 5.1 Long-Term Stability of Lyophilized Compositions Comprising Cyclosporine A

5.1.1 A lyophilized pharmaceutical composition comprising cyclosporine A (5 mg) was prepared according to Example 1 above. The lyophilized composition in form of an almost white, homogeneous, porous lyophilization cake was aliquoted in 6R glass vials, sealed and stored at 25° C. and an air humidity of 60% relative humidity (RH) for a period of 36 months. Aliquots of the material were reconstituted with saline (0.25% (w/v)) to result in a volume 1.25 ml of the reconstituted solution before and after the storage period and the median liposome diameter (Z-average), the polydispersity index as well as the content of cyclosporine A was determined after 3 months, 6 months, 9 months 12 months 18 months, 24 months and 36 months.

5.1.2 It was found that before and after the above-described storage period all parameters were within their respective acceptance criteria. More specifically, the polydispersity index (PI) was lower or equal to 0.50 before and after each storage time period. Furthermore, the median liposome diameter (Z-average) was in the prescribed range of from 40 to 100 nm before and after each storage time period. Furthermore, the CsA content of the reconstituted solution were within the acceptance criteria in the range of from 95.0 to 105.0%.

5.1.3 The long-term stability study as described above was repeated at a temperature of 30° C. and an air humidity of 65% relative humidity (RH). All test parameters as described above were found within their acceptance criteria (as above) before and after a storage period of 3 months, 6 months, 9 months and 12 months.

5.1.4 The long-term stability study as described above was repeated using a lyophilized pharmaceutical composition comprising cyclosporine A (5 mg) prepared according to Example 1 above, wherein however, the lyophilized composition had a content of saccharose necessary to give a liquid composition with a content of saccharose of 7.5 wt.-% with regard to the total amount of the liquid composition after reconstitution.

5.1.5 In this case also, it was found that before and after the above-described storage period all parameters were within their respective acceptance criteria. More specifically, the polydispersity index (PI) was lower or equal to 0.50 before and after each storage time period. Furthermore, the median liposome diameter (Z-average) was in the prescribed range of from 40 to 100 nm before and after each storage time period. Furthermore, the CsA content of the reconstituted solution were within the acceptance criteria in the range of from 95.0 to 105.0%. 5.1.6 The experiments described under items 5.1.1 to 5.1.3 above were repeated using a lyophilized pharmaceutical composition comprising 10 mg of cyclosporine A prepared according to Example 1 above. In this case also, it was found that before and after the above-described storage period all parameters were within their respective acceptance criteria. More specifically, the polydispersity index (PI) was lower or equal to 0.50 before and after each storage time period. Furthermore, the median liposome diameter (Z-average) was in the prescribed range of from 40 to 100 nm before and after each storage time period. Furthermore, the CsA content of the reconstituted solution were within the acceptance criteria in the range of from 95.0 to 105.0%.

### 5.2 Long-Term Stability of Non-Lyophilized Liquid Compositions Comprising Cyclosporine A

5.2.1 A liquid solution of liposomally solubilized cyclosporine A with a CsA concentration of 4 mg/mL in saline (0.25% (w/v) was prepared according to Example 1 above, without however addition of a disaccharide and without intermediate lyophilization. The solution was aliquoted in glass vials as described above sealed and stored at 25° C. and an air humidity of 60% relative humidity (RH).

5.2.1 After 3 months of storage the formation of a precipitate was observed visually as an indicator for physical instability of the liposomal solution.

### Example 6: Nebulization Experiments and Aerosol Characterization

6.1 2.5 ml (corresponding to 10 mg of CsA) of the colloidal solution as prepared in Example 1 were aerosolized by means of a specially adapted electronic vibrating membrane nebuliser of the PARI eFlow 30 XL type having a mixing chamber and breathing in/out valves at a flow rate of 15 L/min according to EUROPEAN PHARMACOPOEIA 7.3; 2.9.44.

6.2 The droplet size distribution of the thus produced aerosol was characterized by laser diffraction using a Malvern MasterSizer X: The mass average particle diameter thus determined was 3.3 μm (Standard Deviation (SD) 0.1) at a geometric standard deviation of 1.5. The respirable particle fraction (RF) <5 μm was 65.3% (SD 2.8), the respirable particle fraction having a particle size <3.3 μm was 37.7% (SD 2.2).

6.3 In an inhalation experiment (adult; flow rate 15 mL/min) a total amount of 9897 μg of ciclosporine A in form of a reconstituted liquid formulation as described in Example 1 above was filled in and administered with the electronic vibrating membrane nebuliser (PARI eFlow 30 XL). The delivered dose (DD) of ciclosporine A was 7339 μg (SD: 471). The respirable dose (RD) <5 μm was 6534 μg (66.0%; SD 4.3%); the RD <3.3 μm was 4461 μg (45.1%; SD 3.2%) and the respirable dose (RD) <2 μm was 1080 μg (10.9%; SD 0.9%).

1. A lyophilized pharmaceutical composition for reconstitution in an aqueous carrier liquid comprising:
a) liposome-forming structures comprising
i. a therapeutically effective amount of cyclosporine A (CsA);
ii. a membrane-forming substance selected from the group of phospholipids; and
iii. a solubility-enhancing substance selected from the group of non-ionic surfactants;
and
b) at least one disaccharide selected from the group consisting of saccharose, lactose and trehalose,
wherein the at least one disaccharide is present in an amount of at least 40 wt.-% with regard to the total weight of the lyophilized composition.

2. The composition according to claim 1, wherein the at least one disaccharide is present in an amount selected in the range of from of 50 wt.-%- to 80 wt.% with regard to the total weight of the lyophilized composition.

3. The composition according to any preceding claim, wherein the liposome forming structures comprise a bilayer membrane formed of the membrane-forming substance selected from the group of phospholipids.

4. The composition according to any preceding claim, wherein the liposome-forming structures are at least partly present in unilamellar form.

5. The composition according to any preceding claim, wherein the liposome-forming structures comprise an inner lumen surrounded or at least partially surrounded by the bilayer membrane formed of the membrane forming substance selected from the group of phospholipids.

6. The composition according to any preceding claim, wherein the inner lumen of the liposome-forming structures is at least partially dehydrated.

7. The composition according to claim 5 or 6, wherein the inner lumen of the liposome-forming structures contains the at least one disaccharide selected from the group consisting of saccharose, lactose and trehalose.

8. The composition according to any preceding claim, wherein the CsA is at least partially incorporated in the bilayer membrane of the liposome-forming structures.

9. The composition according to any preceding claim, wherein the CsA is by at least about 90% or even at least about 95% to about 97.5% incorporated in the bilayer membrane of the liposome-forming structures.

10. The composition according to any preceding claim, wherein the composition comprises cyclosporine A in an amount of from 2 to 4 wt.-%, based on the weight of the lyophilized composition.

11. The composition according to any preceding claim, wherein the at least one disaccharide is saccharose (sucrose).

12. The composition according to any preceding claim, wherein the membrane forming substance selected from the group of phospholipids is a lecithin selected from the group consisting of soy bean lecithin, Lipoid S75, Lipoid S100, Phospholipon®G90, 100 or a comparable lecithin.

13. The composition according to any preceding claim, wherein the content of the membrane-forming substance selected from the group of phospholipids is from about 10 to about 30 wt.-% and preferably from about 20 to about 30 wt.-%, based on the weight of the lyophilized composition.

14. The composition according to any preceding claim, wherein the solubility-enhancing substance selected from the group of non-ionic surfactants is selected from the group of polysorbates.

15. The composition according to any preceding claim, wherein the weight ratio of phospholipid to the solubility enhancing substance selected from the group of non-ionic surfactants, preferably the polysorbate, is selected in the range of from 15:1 to 9:1, preferably between from about 14:1 to about 12:1, for example, about 13:1.

16. The lyophilized pharmaceutical composition of any preceding claim for use as a medicament for pulmonary application.

17. The lyophilized pharmaceutical composition for use according to claim 16, wherein the pulmonary application is carried out after reconstitution of the lyophilized pharmaceutical composition of any of claims 1 to 15 in a sterile aqueous carrier liquid to form a colloidal solution or dispersion.

18. The lyophilized pharmaceutical composition for use according to claim 16 or 17, wherein the pulmonary application is carried out after conversion of the composition into an aerosol, such as by nebulization.

19. The lyophilized pharmaceutical composition for use according to any of claims 16 to 18, wherein the pulmonary application is carried out by inhalation.

20. The lyophilized pharmaceutical composition for use according to any of claims 16 to 19 for the prophylaxis and treatment asthma, refractory asthma, chronic obstructive bronchitis, parenchymal, fibrotic and interstitial lung diseases and inflammations, and preferably for the prevention and treatment of acute and chronic organ transplant rejection reactions after lung transplantations and the diseases resulting therefrom such as bronchiolitis obliterans.

21. A kit for the preparation of an aqueous liposomal dispersion for inhalation comprising a therapeutically effective amount of cyclosporine A in liposomally solubilized form, comprising
a lyophilized pharmaceutical composition according to any of claims 1 to 15, and
an aqueous carrier liquid.

22. The kit according to claim 21, wherein the sterile aqueous carrier liquid is an aqueous sodium chloride solution.

23. A process for the preparation of an aqueous liposomal dispersion for inhalation comprising cyclosporine A in liposomally solubilized form by reconstitution of the lyophilized pharmaceutical composition of any one of claims 1 to 15, comprising dispersing the lyophilized pharmaceutical composition according to any of claims 1 to 15 in a sterile aqueous carrier liquid.

24. A liquid liposomal dispersion comprising an aqueous carrier liquid and a therapeutically effective amount of cyclosporine A in liposomally solubilized form, prepared by a process comprising dispersing the lyophilized pharmaceutical composition of any of claims 1 to 15 in an aqueous carrier liquid.

25. The liquid liposomal dispersion of claim 24, wherein the at least one disaccharide selected from the group consisting of saccharose, lactose and trehalose is present in an amount of 5 to 15 wt.-%, based on the total weight of the liquid liposomal dispersion.

26. The liquid liposomal dispersion according to claim 24 or 25, wherein the dispersion comprises liposomes with a z-average diameter as measured by photon correlation spectroscopy in the range of from about 40 to about 100 nm.

27. The liquid liposomal dispersion according to any of claims 24 to 26, wherein the dispersion comprises liposomes with a n the z-average diameter as measured by photon correlation spectroscopy which is equal or up to 20% larger than the z-average diameter of the liposomes used to prepare the lyophilized pharmaceutical composition of any of claims 1 to 15 prior to lyophilization, preferably which is equal or up to 20% larger than the liposomes formed by a process according to claim 28 before lyophilization.

28. A process for the preparation of a lyophilized pharmaceutical composition comprising a therapeutically effective amount of cyclosporine A in liposomally solubilized form for reconstitution in an aqueous carrier liquid, preferably for the preparation of the lyophilized pharmaceutical composition of any of claims 1 to 15, the process comprising the steps:
(a) providing a liquid aqueous dispersion of liposomes, said liposomes comprising:
i. a therapeutically effective amount of cyclosporine A (CsA);
ii. a membrane-forming substance selected from the group of phospholipids; and
iii. a solubility-enhancing substance selected from the group of non-ionic surfactants; and optionally
iv. one or more further excipients, such as buffers and/or chelating agents;
wherein said liquid aqueous dispersion further comprises at least one disaccharide disaccharide selected from the group consisting of saccharose, lactose and trehalose, which is present in an amount of at least 40 wt.-% with regard to the total weight of the lyophilized composition; and
(b) lyophilizing said aqueous dispersion.

29. The process according to claim 28, comprising the steps:
(a1) providing an aqueous mixture comprising:
at least one disaccharide selected from the group consisting of saccharose, lactose and trehalose;
a membrane-forming substance selected from the group of phospholipids;
a therapeutically effective amount of cyclosporine A (CsA); and
a solubility-enhancing substance selected from the group of non-ionic surfactants; and optionally
one or more further excipients
and
(a2) exposing the aqueous mixture to homogenization conditions; and
(b1) lyophilizing the resulting homogenized mixture to form the lyophilized pharmaceutical composition.

30. A lyophilized pharmaceutical composition obtainable by a process comprising the steps of
(a) providing a liquid aqueous dispersion of liposomes, said liposomes comprising
i. a therapeutically effective amount of cyclosporine A (CsA);
ii. a membrane-forming substance selected from the group of phospholipids; and
iii. a solubility-enhancing substance selected from the group of non-ionic surfactants; and optionally
iv. one or more further excipients, such as buffers and/or chelating agents.
wherein said liquid aqueous dispersion further comprises at least one disaccharide selected from the group consisting of saccharose, lactose and trehalose, which is present in an amount of at least 40 wt.-% with regard to the total weight of the lyophilized composition; and
(b) lyophilizing said aqueous dispersion.
